(19) United States
(12) Patent Application Publication
(10) Pub. No.: US 2024/0277966 A1
(43) Pub. Date: Aug. 22, 2024

(54) A RESPIRATORY SUPPORT APPARATUS

(71) Applicant: Fisher & Paykel Healthcare Limited, Auckland (NZ)

(72) Inventors: David M. Rapoport, New York, NY (US); Kevin Blake Powell, Auckland (NZ); Laurence Gulliver, Auckland (NZ); Andre Van Schalkwyk, Auckland (NZ); Kevin Peter O'Donnell, Auckland (NZ)

(21) Appl. No.: 17/997,230

(22) PCT Filed: Apr. 30, 2021
(86) PCT No.: PCT/NZ2021/050074

§ 371 Date: May 8, 2023

Related U.S. Application Data
(60) Provisional application No. 63/018,807, filed on May 1, 2020.
(60) Provisional application No. 63/029,185, filed on May 22, 2020.

(51) Int. Cl. A61M 16/20; A61M 16/00; A61M 16/08; A61M 16/10; A61M 16/12; A61M 16/16; A61M 16/22

(52) U.S. Cl. CPC A61M 16/209; A61M 16/0066; A61M 16/0096; A61M 16/0891; A61M 16/101; A61M 16/1065; A61M 16/125; A61M 16/16; A61M 16/22; A61M 2205/3334

(57) ABSTRACT

Embodiments of a respiratory support apparatus are disclosed comprising features configured to minimize, reduce or contain aerosols carrying pathogens that can cause diseases such as COVID 19, SARS, MERS, Tuberculosis, or any other infectious diseases. Embodiments of a respiratory support apparatus are also provided configured to at least reduce the amount of oxygen required, during use of the apparatus, from an external oxygen supply such as an oxygen tank or hospital wall supply. Embodiments of such apparatus are provided with means to recirculate expiratory gases, and/or redirect leak flow. Embodiments of such apparatus are provided in which expiratory gases are sucked away from the patient. Embodiments of such apparatus are provided comprising a first flow generator for delivering inspiratory gases, and a second flow generator for removing expiratory gases.

The present disclosure relates to a respiratory support apparatus comprising a flow generator configured to generate a gases flow for inspiration by a patient. International Application No. PCT/NZ2017/050119, titled “Thermistor Flow Sensor Having Multiple Temperature Points”, filed on Sep. 13, 2017, International Application No. PCT/NZ2016/050193, titled “Flow Path Sensing for Respiratory support Apparatus”, filed on Dec. 2, 2016, International Application No. PCT/IB2016/053761, titled “Breathing Assistance Apparatus”, filed on Jun. 24, 2016, and PCT/NZ2016/050101 filed on Jun. 27 2016, titled “Exhalation Port”, International Application No. PCT/NZ2013/000166 filed Sep. 9 2013, are hereby incorporated by reference in their entireties. This application claims priority from provisional applications U.S. 63/018,807 filed 1 May 2020 and U.S. 63/029,185 filed on 22 May 2020, both of which are hereby incorporated by reference in their entireties.

### FIELD OF THE DISCLOSURE

### Background of the Disclosure

Respiratory support apparatuses are used in various environments such as hospitals, medical facilities, residential care, or home environments to deliver a gases flow to users or patients. Respiratory support apparatus comprise a flow generator to generate a gases flow. The gases flow is delivered to the patient via an inspiratory conduit and a patient interface, the patient interface being mounted on the head of the patient, for example using headgear. Examples of such respiratory support apparatus include CPAP, PEEP, Bi-level and High Flow apparatus. Some respiratory support apparatus are configured to selectively deliver more than one type of respiratory therapy, for example CPAP and High Flow therapies.

### SUMMARY OF THE DISCLOSURE

One example of a respiratory support apparatus is a high flow respiratory support apparatus, to deliver a high flow of heated, and typically humidified, gases to a patient. The gases are delivered via an unsealed patient interface. Examples of such a patient interface are a nasal cannula or a tracheostomy interface for example. A typical high flow apparatus comprises a nasal high flow apparatus configured to provide nasal high flow respiratory support (NHF therapy). The nasal cannula may comprise one or more prongs configured to be received in the nares of the patient. The prongs may be configured not to seal against the nares, such that gases, including exhalation gases, can leak around the prongs. If a tracheostomy interface is provided, one or more leak openings are typically provided to allow leaking of expiratory gases.

Another example of a respiratory support apparatus is a CPAP/BiLevel apparatus being a pressure controlled apparatus that attempts to deliver a set pressure. Set pressure may be a constant pressure (CPAP) or may be BiLevel pressure where the two pressures are used, for example, one for inspiration, the other for expiration (noting that in BiLevel therapy the different pressures are not necessarily synchronized with patient inspiration and expiration). Almost all present systems designed to deliver CPAP/BiLevel have a deliberate leak port either in a vented mask or placed near or in the circuit to a non-vented mask, which mask has as a purpose to allow venting of exhaled gases.

It has been found that pathogens, microbes and/or or other disease carrying matter, and/or non-pathogenic substances or contaminants, such as medicaments, can be present in the expiratory gases generated by a patient when exhaling. In some cases, such matter comprises aerosols. Aerosols, being a suspension of tiny particles or droplets in the air, can contain substances and/or contaminants that are undesirable for persons to come in contact with. For a patient suffering from infectious diseases e.g. COVID 19, SARS, MERS, Tuberculosis, or any other infectious diseases, the aerosols can carry pathogens that can cause these diseases. The exhaled gases, including any aerosols, can present a risk of carrying and spreading these diseases. Such aerosols can also carry other substances, such as medicaments, which are also typically undesirable to disperse to anyone other than the patient to which they have been administered. Both NHF and CPAP/Bilevel circuits, as currently in use, have high levels of gas flow exiting from the circuit, that contain or mix with gas exhaled from the patient. These gas flows are vented from the circuit and thus can increase the risk of contamination by carrying the disease. Preventing the dispersion of infectious aerosols is needed to reduce transmission of infectious diseases. However, the source of overflow of contaminated gases differs in NHF and CPAP/bilevel, but in both treatment devices carries the same consequence. In NHF, the flow delivered to the patient is high (>20 l/min) and in excess of that inhaled during each breath. During exhalation, this high flow entrains the patient's exhalation through the nose around a non-sealing nasal prong. Furthermore, this high flow may generate aerosols from the nose independent of breathing. In CPAP/bilevel, the excess flow delivered to the patient needs only be enough to maintain pressure in the (sealed) mask and circuit, but a leak must be inserted in the circuit to allow exhaled gases from the patient to exit prior to each inhalation to prevent rebreathing of CO2.

An additional limitation of existing NHF and CPAP/Bilevel systems, is that for the systems to function, a relatively high flow of gas must be delivered to the patient in excess of what is breathed by the patient. It can b difficult to raise the oxygen concentration without adding large amounts of supplemental oxygen to the circuit. This potentially wasteful addition of oxygen may limit use of these devices when using oxygen from a portable tank or in circumstances where unlimited wall pressure of oxygen is not available.

Another problem is that the oxygen supply in a hospital is a limited commodity which has a cost associated with it, as well as a practical limit as to how much oxygen can be stored on site, or supplied to the hospital. At times of high demand, hospitals can struggle to meet the oxygen demands created by the increase in patients requiring respiratory care, for example due to widespread respiratory diseases such as Covid-19.

The present disclosure relates to a respiratory support apparatus that addresses any one or more of these two limitations for one or both of a high flow or pressure therapy apparatus, for example a NHF or CPAP/bilevel apparatus.

Aspects of this disclosure address one or more of these limitations, or otherwise provide an improved or alternative apparatus, by 1) capturing and containing some or all of the overflow gases that would normally be vented to the environment, and 2) recirculating some or all of these gases to the flow generator so as to conserve oxygen. The present disclosure also relates to a respiratory support apparatus that helps to reduce or minimize oxygen wastage. The apparatus can help with oxygen conservation during provision of respiratory support. The apparatus may be primarily configured to capture exhaled gases and recirculate the oxygen, thereby conserving oxygen and reducing overall oxygen usage. This can have the added benefit of capturing exhaled aerosols.

In accordance with this disclosure we provide a respiratory support apparatus configured to deliver pressure therapy (such as CPAP or Bilevel therapy), which:
a. captures and contains some or all of the overflow gases that would normally be vented to the environment; and/or
b. recirculates some or all of these gases to the flow generator so as to conserve oxygen; and/or
c. reduces or minimizes oxygen wastage.

In accordance with this disclosure we provide a respiratory support apparatus configured to deliver high flow therapy (such as NHF therapy), which:
a. captures and contains some or all of the overflow gases that would normally be vented to the environment; and/or
b. recirculates some or all of these gases to the flow generator so as to conserve oxygen; and/or
c. reduces or minimizes oxygen wastage.

In accordance with this disclosure we provide a respiratory support apparatus configured to deliver an inspiratory gases flow to a patient, which:
a. captures and contains some or all of the overflow gases that would normally be vented to the environment; and/or
b. recirculates some or all of these gases to the flow generator so as to conserve oxygen; and/or
c. reduces or minimizes oxygen wastage.

In accordance with aspects of this disclosure we provide a single apparatus which can be used to deliver pressure therapy (such as CPAP or Bilevel therapy) or high flow therapy (such as NHF) via a flow generator of the apparatus. The apparatus comprises a controller provided with, or configured to be operative according to, control logic or software that can control the flow generator based on feedback from one or more sensors (such as any one or more of a flow sensor, pressure sensor, flow generator motor speed sensor) to provide pressure or high flow therapy. The apparatus can be converted to provide one or other of these therapies depending on the components attached to the apparatus.

For example, the apparatus could be configured to be connected to a common inspiratory gases delivery conduit, with a kit of different patient interfaces being provided for connection to the conduit. For example, the same inspiratory conduit could be used with sealed interfaces for pressure therapy such as CPAP or Bilevel therapy, or high flow therapy. Example sealed interfaces include a full face mask, a nasal mask, or sealed nasal prongs such as nasal pillows. Example unsealed interfaces include a tracheostomy interfaces or a unsealed nasal cannula. The apparatus may be configured such that a user can select a mode of operation of the apparatus, each mode of operation relating to a type of therapy. The user can couple the specific interface to the apparatus in accordance with the mode of operation selected, and the flow generator may then be controlled to generate a user set pressure (CPAP) or cycle between user set IPAP and EPAP; or control the flow generator to a user set flow rate (high flow therapy).

In accordance with certain features, aspects and advantages of a first embodiment disclosed herein, a respiratory support apparatus configured to provide a gases flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator outlet to deliver the gases flow to the patient;
   an expiratory conduit configured to extend between, and be in fluid communication with, a region at or adjacent the patient interface and the flow generator inlet, to provide a flow path for expiratory gases between the region at or adjacent the patient interface and the flow generator inlet;
wherein the generation by the flow generator of the gases flow also forces expiratory gases along the expiratory conduit from the region at or adjacent the patient interface; and
wherein the apparatus comprises a leak port or valve configured to provide a controlled leak of the gases flow between the flow generator outlet and the patient interface.

The expiratory gases may be drawn along the expiratory conduit via suction forces generated by the flow generator in the expiratory conduit. The expiratory gases may be pushed along the expiratory conduit via pressure forces generated in the patient interface by the delivery of the gases flow to the patient.

The leak port or valve may be provided at the flow generator outlet, upstream of the patient interface, or at the patient interface.

The leak port or valve may be configured to leak between 5 and 50% of the high flow of gases, in some examples between 15 and 40%, and in other examples around 30%.

The leak port or valve may be configured to leak a predetermined flow rate of the gases flow.

The leak port or valve may be configured to leak a flow rate of the gases flow between 15 l/min and 25 l/min.

The leak port or valve may be configured to leak a predetermined proportion of the flow rate of the gases flow.

The leak port or valve may be configured to vary the amount of the gases flow that leaks through the leak port or valve.

The leak port may be configured to leak a predetermined amount of flow or a predetermined proportion of the flow rate of gases such that flow rate or amount of gases delivered to the patient interface are less than the flow rate or amount of gases generated by the flow generator.

The flow rate of gases drawn into the expiratory conduit or the total amount of gases drawn into the expiratory conduit may be more than the flow rate or amount of gases delivered to the patient via the interface. This is advantageous because the gases drawn away from the patient is greater than the gases provided to the patient, thereby making it more likely that most or all the exhaled gases are drawn away from the patient rather than being vented to ambient. This reduces the chances or any infectious diseases within the exhaled gases from being transmitted into ambient and possibly infecting others.

The flow generator inlet may comprise an ambient inlet configured to receive ambient air and/or a supplemental gases inlet configured to receive supplemental gases. The supplemental gases may comprise concentrated oxygen.

The flow generator may be configured to recirculate expiratory gases received from the expiratory conduit into the gases flow.

A filter may be provided between the patient interface and the flow generator, configured to filter the expiratory gases. The filter may be a HEPA and/or EPA filter.

A gases scrubber may be provided between the patient interface and the flow generator, configured to scrub the expiratory gases. The gases scrubber may be a CO2 scrubber.

The respiratory support apparatus may comprise an inspiratory conduit configured to provide an inspiratory gases flow path for the gases flow between the flow generator outlet and the patient interface.

The respiratory support apparatus may comprise a humidifier, configured to humidify the high flow of gases.

The leak port or valve may be upstream of the humidifier.

The leak port or valve is downstream of the humidifier.

A plurality of leak ports or valves may be provided.

The patient interface may comprise a full face mask configured to seal around only the nose and mouth of the patient, a total face mask configured to seal around the eyes, nose and mouth of the patient, or a total head mask that seals around the eyes, nose and mouth of the patient, and additionally seals around at least part of the top and/or rear of the head of the patient. In other examples the patient interface may be a non-sealing interface, such as a nasal cannula having one or more non-sealing prongs for example.

In some examples, a face shield may be provided in addition to the patient interface. The face shield could be considered a second patient interface. The face shield is configured to cover at least the nose and mouth of the patient in the sense that the face shield directs expiratory gases from the nose and/or mouth in a given direction, for example towards the leak port or valve/The face shield in some examples does not seal against the patient's face. The face shield is preferably shaped to direct expiratory gases from the patient in a given direction, for example via the face shield comprising one or more inclined shield walls.

In some examples the face shield comprises any of:
a) a full face mask that covers the nose and mouth of the patient;
b) a total face mask that covers the nose, mouth and eyes of the patient;
c) a dome that fits over the entire head of the patient and is secured at the neck of the patient;
d) a hood that covers the head and neck, the hood comprising a skirt that extends around, but does not seal with, the neck of the patient.

The flow generator may comprise a blower. The blower may comprise a plurality of blowers. One of the blowers of the plurality of blowers may be configured to generate the gases flow, and wherein another of the blowers of the plurality of blowers is configured to force expiratory gases along the expiratory conduit, for example via generation of suction force in the expiratory conduit.

The flow rate of expiratory gases generated by the flow generator in the expiratory conduit may substantially equal the flow rate of the flow of gases generated by the flow generator, such that the respiratory support apparatus functions as a closed system, for example where the apparatus comprises a pressure support apparatus.

The flow rate of expiratory gases generated by flow generator, in the expiratory conduit may substantially greater than the flow rate of the flow of gases generated by the or each blower, such that the respiratory support apparatus functions as a closed system, for example where the apparatus comprises a high flow apparatus.

In a high flow respiratory support apparatus the flow rate of the expiratory gases generated by the suction forces generated by the flow generator must be larger than the flow rate of the sum of all the gases delivered to the patient. The expiratory flow will capture all of the gases delivered in the inspiratory side of the circuit. Further the expiratory flow i.e. flow in the expiratory conduit due to the suction from the blower will also draw in (i.e. pull in) room air (i.e. ambient air) from around the patient's face (and around the face shield if provided) The expiratory flow is augmented by air from the room pulled in from around the patient's face (and around a face shield if provided).

The face shield is an unsealed interface that forms a receptacle to direct exhaled gases from the patient toward the expiratory conduit. The face shield (i.e. face cover) comprises one or more walls that form a receptacle to direct exhaled gases toward the expiratory conduit.

In a pressure support respiratory apparatus, such as a CPAP/bilevel application, the expiratory flow may be less than or equal to the inspiratory flow out the blower, but be augmented at the blower inlet by gas external to the circuit. In one example implementation for the pressure support respiratory apparatus, the expiratory flow in the expiratory conduit may be less than the inspiratory flow out of the blower due to patient exhalation being less than the inspiratory flow from the blower.

The patient interface may comprise:
a) a sealed nasal mask configured to deliver the gases flow to the nose of the patient;
b) a sealed oral mask configured to deliver the gases flow to the mouth of the patient;
c) a sealed full face mask configured to deliver the gases flow to the nose and mouth of the patient;
d) a tracheostomy interface or tracheostomy tube;
e) a nasal cannula comprising at least one prong configured to be received in a nare of the patient, wherein the at least one prong is a non sealing prong;
f) a nasal pillow configured to seal against the nare of the patient.

Where a face shield or second patient interface is provided, the first and second patient interfaces may be integral so as to form a single component.

According to another aspect of this disclosure there is provided a respiratory system comprising:
a) the respiratory support apparatus of any one of the above statements;
b) a humidifier; and
c) an inspiratory conduit.

According to a further aspect of this disclosure there is provided a respiratory support apparatus configured to provide a gases flow to a patient, the respiratory support apparatus comprising:
a flow generator configured to generate the gases flow and comprising an inlet and an outlet,
   an inspiratory conduit configured to be connected to the flow generator outlet via an outlet connection to deliver the gases flow to the patient;
an expiratory conduit configured to be connected to the flow generator inlet via an inlet connection to deliver expiratory gases from the patient;
wherein the flow generator, inlet connection and expiratory conduit are configured to be pneumatically coupled such that the flow generator generates a suction force in the inlet connection and expiratory conduit to suck expiratory gases from the patient along the expiratory conduit;
wherein the apparatus comprises a leak port or valve configured to provide a controlled leak of the gases flow downstream of the flow generator outlet.

According to a further aspect of this disclosure there is provided a respiratory support apparatus configured to provide a gases flow to a patient via a patient interface, the respiratory support apparatus comprising:
a flow generator configured to generate the gases flow;
an expiratory conduit configured to receive expiratory gases from the patient interface;
wherein the flow generator and the expiratory conduit are configured to be pneumatically coupled such that the flow generator generates a force in the expiratory conduit to force expiratory gases from the patient along the expiratory conduit.

The force may be a suction force.

The apparatus may comprise a leak port or valve configured to provide a controlled leak of the gases flow downstream of the flow generator.

According to a further aspect of this disclosure there is provided a high flow respiratory support apparatus configured to provide a gases high flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a first patient interface configured to be mounted on the patient and to receive the gases flow from the flow generator outlet;
a second patient interface arranged to cover at least the nose and mouth of the patient;
an expiratory conduit configured to extend between, and be in fluid communication with, the second patient interface and the flow generator inlet, to provide a flow path for expiratory gases between the second patient interface and the flow generator inlet;
wherein the generation by the flow generator of the gases flow also generates suction in the expiratory conduit, such that the flow generator draws expiratory gases along the expiratory conduit from inside the second patient interface; and
wherein the apparatus comprises a leak port or valve configured to provide a controlled leak of the gases flow between the flow generator outlet and the first patient interface.

According to another aspect of this disclosure there is provided a respiratory pressure support apparatus configured to provide a gases flow to a patient, the apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator outlet; the patient interface comprising a seal arranged to seal around at least the nose and mouth of the patient;
an expiratory conduit configured to extend between, and be in fluid communication with, the patient interface and the flow generator inlet, to provide a flow path for expiratory gases between the patient interface and the flow generator inlet;
wherein the generation by the flow generator of the gases flow also generates forces in the expiratory conduit, such that the flow generator forces expiratory gases along the expiratory conduit from inside the patient interface; and
wherein the apparatus comprises a leak port or valve configured to provide a controlled leak of the gases flow between the flow generator outlet and the patient interface.

The respiratory pressure support apparatus may be any one or more of a:
a) CPAP apparatus;
b) Bilevel apparatus;
c) PEEP apparatus.

According to a further aspect of this disclosure there is provided a respiratory support apparatus configured to provide a gases flow to a patient comprising:
a flow generator configured to generate the gases flow and comprising an inlet and an outlet, the flow generator being configured to draw inspiratory gases in through the inlet, and to deliver the gases flow through the outlet;
a breathing circuit in fluid communication with the outlet of the flow generator, patient interface, and the inlet of the flow generator, wherein the breathing circuit defines a closed gases flow path to deliver gases generated by the flow generator to the patient interface, and to recirculate expired gases exhaled from the patient interface, from the patient interface to the inlet of the flow generator.

The breathing circuit may comprise an inspiratory conduit extending between the flow generator outlet and the patient, and an expiratory conduit extending between the patient and the flow generator inlet.

The flow generator may generate a gases flow at a pressure to deliver gases to the patient interface.

The expiratory conduit preferably pneumatically couples the patient interface to the flow generator inlet such that the flow generator creates suction within the expiratory conduit to draw gases away from the patient interface.

The breathing circuit may comprise a plurality of patient interfaces, the plurality of patient interfaces comprising an unsealed patient interface that couples to the inspiratory conduit and a second patient interface that covers at least the nose and mouth of the patient and which is pneumatically coupled to the expiratory conduit.

The apparatus may be a high flow apparatus.

According to a further aspect of this disclosure there is provided a method of providing respiratory support to a patient using a respiratory support system comprising a flow generator and a patient interface, the method comprising steps of:
controlling the flow generator to provide an inspiratory gases flow to the patient interface;
controlling the flow generator to force expiratory gases from the patient interface, or from a region at or adjacent the patient interface;
leaking some of the inspiratory gases flow; and
recirculating the expiratory gases to the patient.

According to another aspect of this disclosure there is provided a high flow respiratory support apparatus configured to provide a gases high flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient, the patient interface comprising an exhalation port through which expiratory gases from the patient can flow; wherein the exhalation port is pneumatically coupled to the flow generator such that the flow generator creates a negative pressure at the patient sufficient to suck expiratory gases through the exhalation port; the flow rate of expiratory gases sucked through the exhalation port being equal to or greater than the flow rate of the gases flow delivered to the patient.

According to a further aspect of this disclosure there is provided a respiratory pressure support apparatus configured to provide a gases flow to a patient, the apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator outlet;
the patient interface comprising an exhalation port;
the exhalation port being pneumatically coupled to the flow generator such that the flow generator creates forces at the patient sufficient to force expiratory gases through the exhalation port.

According to a further aspect of this disclosure there is provided a respiratory support apparatus configured to provide a gases flow to a patient, the apparatus comprising:
a flow generator configured to generate the gases flow and comprising an inlet and an outlet, the flow generator being configured to draw inspiratory gases in through the inlet, and to deliver the gases flow through the outlet;
an expiratory circuit configured to receive expiratory gases from the patient;
the expiratory circuit being pneumatically coupled to the flow generator inlet such that expiratory gases from the expiratory circuit are received in the flow generator inlet and recirculated into the gases flow delivered to the patient.

CO₂ may be scrubbed from the expiratory gases prior to being recirculated to the patient.

According to a further aspect of this disclosure there is provided an expiratory circuit for use in a respiratory support apparatus, the expiratory circuit comprising:
an expiratory conduit configured to be connected between a patient interface and a flow generator inlet of the respiratory support apparatus.

The expiratory circuit may comprise any one or more of:
a) a CO₂ scrubber;
b) a filter;
c) an adaptor configured to pneumatically couple the expiratory conduit to an inlet of the flow generator of the respiratory support apparatus.
d) one or more conduit connectors
e) a disinfection unit configured to disinfect gases passing through the expiratory conduit, wherein the disinfection unit is located upstream of the flow generator inlet.

According to a further aspect of this disclosure there is provided a respiratory support apparatus configured to provide a gases flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a first patient interface configured to be mounted on the patient and to receive the gases flow from the flow generator outlet to deliver the gases flow to the patient;
a second patient interface being configured to cover at least the nose and mouth of the patient;
an expiratory conduit configured to extend between, and be in fluid communication with, the patient interface and the flow generator inlet, to provide a flow path for expiratory gases between the patient interface and the flow generator inlet;
wherein the generation by the flow generator of the gases flow also generates forces in the expiratory conduit, such that the flow generator forces expiratory gases along the expiratory conduit from the patient interface; and
wherein the apparatus comprises a leak port or valve configured to provide a controlled leak of the gases flow between the flow generator outlet and the patient interface.

According to a further aspect of this disclosure there is provided a respiratory support apparatus comprising
a flow generator configured to generate a flow of gases,
   the flow generator further comprising an inlet and an outlet;
   a patient interface in fluid communication with the patient airways,
   a gases delivery circuit coupled to the outlet of the flow generator and the patient interface,
   a recirculation circuit in fluid communication with the patient interface, the recirculation circuit coupled to the inlet of the flow generator,
   the flow generator creating forces within the recirculation circuit such that exhaled gases are directed into the recirculation circuit and the exhaled gases from the patient are recirculated.

The gases delivery circuit may comprise a leak port to vent out some of the gases generated by the flow generator.

The gases delivery circuit may comprise a humidifier.

The patient interface may be an unsealed interface, and the apparatus comprises a second patient interface that extends over at least the nose and mouth of the patient, the second patient interface positioned on the patient using headgear, and the patient interface is positioned inside the second patient interface.

The recirculation circuit may comprise a recirculation conduit that is coupled to the second patient interface.

The flow generator may be flow controlled flow generator that is configured to deliver a set flow rate.

The patient interface may form a seal with the patient's face or a seal with a patient's airway.

The apparatus may comprise a leak port formed within the patient interface or located adjacent the patient interface, and the recirculation circuit is in fluid communication with the leak port to capture and direct exhaled gases from the patient toward the flow generator.

The gases delivery circuit may comprise a humidifier.

The flow generator may be a pressure controlled flow generator, wherein the flow generator is controlled to deliver a set pressure.

The recirculation circuit may comprise a recirculation conduit and a scrubber within the circuit, wherein the scrubber is configured to remove or reduce CO₂ within the recirculated gases and/or the recirculation circuit comprises a filter, wherein the filter is located upstream of the inlet of the flow generator and filters recirculated gases prior to entering the flow generator inlet.

The recirculation circuit may comprise a disinfection unit, wherein the disinfection unit is configured to disinfect recirculated gases.

According to another aspect of this disclosure there is provided a respiratory support apparatus configured to provide a gases flow to a patient, the respiratory support apparatus comprising:
at least one flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator outlet to deliver the gases flow to the patient;
an expiratory conduit configured to extend between, and be in fluid communication with, a region at or adjacent the patient interface and the flow generator inlet of the at least one, or a, flow generator, to provide a flow path for expiratory gases between the region at or adjacent the patient interface and the flow generator inlet;
wherein the apparatus is configured to generate forces in the expiratory conduit, such that expiratory gases are forced along the expiratory conduit from the region at or adjacent the patient interface; and
wherein the apparatus comprises a leak port or valve configured to provide a controlled leak of the gases flow between the flow generator outlet and the patient interface;
the apparatus further comprising a leak conduit fluidly coupled between the leak port or valve and the patient interface, and configured to deliver the controlled leak of the gases flow to the patient interface.

The apparatus may comprise a leak manifold at the patient interface and fluidly coupled to the leak conduit, the leak manifold comprising a leak outlet configured to deliver the leak flow to a position adjacent the nose and/or mouth of the patient.

According to a further aspect of this disclosure there is provided a respiratory support apparatus configured to provide a gases flow to a patient, the respiratory support apparatus comprising:
at least one flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator outlet to deliver the gases flow to the patient;
an expiratory conduit configured to extend between, and be in fluid communication with, a region at or adjacent the patient interface and the flow generator inlet of the at least one, or a, flow generator, to provide a flow path for expiratory gases between the region at or adjacent the patient interface and the flow generator inlet;
wherein the apparatus is configured to generate forces in the expiratory conduit, such that expiratory gases are forced along the expiratory conduit from the region at or adjacent the patient interface; and
wherein the apparatus comprises a leak port or valve configured to provide a controlled leak of the gases flow between the flow generator outlet and the patient interface;
the leak port or valve being provided on, and/or shaped and/or positioned to be in fluid communication with, the patient interface.

The leak port or valve may be provided on a leak manifold configured to be fluidly coupled to the patient interface.

The leak port or valve may comprise an array of leak ports.

The expiratory conduit/recirculation conduit may comprise an inlet end, the apparatus comprising a conduit mount configured to mount the inlet end adjacent the patient's face.

The mount may be configured to mount the inlet end adjacent the patient's nose, the patient's mouth, or adjacent both of the patient's nose and mouth.

The mount may comprise any one or more of:
a. a lanyard that is hung around the patient's neck;
b. a neck strap or collar that extends around the patient's neck;
c. a headgear clip or connector, configured to mount the inlet end to a headgear strap or buckle of headgear of the apparatus;
d. a clothing clip configured to be mounted on clothing of the patient;
e. a patient interface clip configured to mount the inlet end on the patient interface.
f. one or more outlet ports or connectors of the patient interface configured to be fluidly coupled with the inlet end of the expiratory conduit.

The expiratory conduit/recirculation conduit and the inspiratory conduit may be configured such that:
a. both conduits are coaxial; or
b. the conduits are secured together such that their longitudinal axes are parallel.

The apparatus may comprise an oxygen concentrator.

The oxygen concentrator may be upstream of the flow generator.

According to a further aspect of this disclosure there is provided a circuit for use as a recirculation circuit with a single limb respiratory support apparatus, wherein the circuit comprises a conduit and a filter, wherein the circuit is coupled between the patient interface or leak port and a flow generator of the respiratory support apparatus to convert a single limb respiratory support apparatus into a dual limb, closed system wherein exhaled gases are recirculated.

The circuit may comprise a scrubber configured to scrub CO₂ from exhaled gases.

The circuit may comprise a disinfection unit to disinfect exhaled gases that are recirculated.

An outlet of the conduit of the circuit may couple to an inlet of the flow generator of the respiratory and an inlet of the conduit couples to a leak port or a patient interface to create a recirculation system, wherein the flow generator creates suction within the circuit.

The respiratory support apparatus may comprise a high flow support apparatus or a pressure support apparatus.

According to a further aspect, the present disclosure relates to a high flow respiratory support apparatus comprising:
a flow generator comprising an inlet and an outlet, the flow generator configured to generate a flow of gases, wherein the flow generator is flow controlled,
an inspiratory gas flow path in fluid communication with the outlet of the flow generator,
   the inspiratory gas flow path comprising an inspiratory conduit and a leak port, the inspiratory conduit and the leak port being fluidically coupled to each other,
a first patient interface in fluid communication with the inspiratory gas flow path, the first patient interface in fluid communication with a patient's nares and the first patient interface engaging the patient's nares in a non sealing manner,
an expiratory gas flow path in fluid communication with the inlet of the flow generator, the expiratory gas flow path comprising an expiratory conduit,
a second patient interface positioned on the patient, the second patient interface defining a cover that encompasses the first patient interface, wherein the second patient interface in fluid communication with the expiratory gas flow path,
wherein the flow generator creates a positive pressure within the inspiratory gas flow path to create a positive flow of gases toward the patient, a portion of the positive flow of gases is leaked out through the leak port such that the total flow or flow rate from the first interface to the patient is less than the flow rate or total flow generated by the flow generator,
wherein the a negative pressure or suction is created within the expiratory gas flow path when the flow generator is generating the flow of gases, and wherein the flow rate or total flow within the expiratory gas flow path is greater than the flow rate or total flow from the first patient interface, and;
wherein the first patient interface and second patient interface are both unsealed.

The high flow respiratory support apparatus wherein the exhaled gases are recirculated through the expiratory gas flow path

The expiratory gas flow path comprises a scrubber, the scrubber configured to scrub CO₂ and/or other gases from the gases flow within the expiratory conduit prior to the gases flow re-entering the inlet of the blower. Optionally the expiratory gas flow path comprises a filter (e.g. a HEPA filter) upstream of the inlet of the flow generator. Optionally the expiratory gas flow path comprises a disinfection unit configured to disinfect the gases flow in the expiratory gases flow path.

The flow generator may comprise a blower. The inspiratory gases flow path may comprise a humidifier configured to humidify the gases from the flow generator prior to being delivered to the patient via the first patient interface. The first patient interface is a nasal cannula comprising a pair of nasal prongs that engage the nares of the patient in an unsealed manner. The second patient interface comprises a face shield or a cover that engages the patient in an unsealed manner.

The high flow respiratory support apparatus is configured to conserve oxygen due to recirculation of the gases. The high flow respiratory support apparatus is further configured to reduce escape of aerosols or infectious diseases within the exhaled gases as the second interface directs exhaled gases (including aerosols) toward the expiratory conduit and the negative pressure within the expiratory conduit draws exhaled gases away from the patient and into the expiratory conduit for recirculation. The recirculation is advantageous as it reduces exhaled gases from escaping to room air (i.e. ambient air).

In one example the apparatus comprises a controller, the controller configured to control the flow generator to be flow controlled such that the flow generator is controlled to generate a set flow rate.

One end of the expiratory conduit is coupled to an outlet port within the second patient interface and an opposing end is coupled to the inlet of the flow generator.

According to a further aspect, the present disclosure related to a pressure support respiratory apparatus comprising:
a flow generator comprising an inlet and an outlet, the flow generator configured to generate a flow of gases, wherein the flow generator is pressure controlled, an inspiratory gas flow path in fluid communication with the outlet of the flow generator,
the inspiratory gas flow path comprising an inspiratory conduit and a leak port, the inspiratory conduit and the leak port being fluidically coupled to each other,
a sealed patient interface in fluid communication with the inspiratory gas flow path, the sealed patient interface providing a flow of gases to the patient, wherein the sealed patient interface creating a pressure within a patient's airways,
an expiratory gas flow path in fluid communication with the leak port and the expiratory gas flow path in fluid communication with an inlet of the flow generator, wherein the expiratory gas flow path comprises an expiratory conduit,
   wherein the flow generator creates a positive pressure within the inspiratory gas flow path to create a positive flow of gases toward the patient,
wherein the a negative pressure or suction is created within the expiratory gas flow path when the flow generator is generating the flow of gases, and wherein the negative pressure draws exhaled gases out through the leak port,
wherein the inspiratory gas flow path, the patient interface and the expiratory gas flow path form a closed flow path (i.e. a sealed gas flow path) with minimal or no leak of gases.

The exhaled gases may be recirculated from the patient interface via the expiratory gas flow path, through the flow generator to the inspiratory gas flow path.

The expiratory gas flow path may comprise a scrubber, the scrubber configured to scrub CO₂ and/or other gases from the gases flow within the expiratory conduit prior to the gases flow re-entering the inlet of the blower. Optionally the expiratory gas flow path may comprise a filter (e.g. a HEPA filter) upstream of the inlet of the flow generator. Optionally the expiratory gas flow path may comprise a disinfection unit configured to disinfect the gases flow in the expiratory gases flow path.

The flow generator may comprise a blower. The inspiratory gases flow path may comprise a humidifier configured to humidify the gases from the flow generator prior to being delivered to the patient via the first patient interface.

The sealed patient interface may be a nasal mask or a full face mask or a total face mask (that seals about the nose, mouth and eyes) or a sealed helmet. The leak port may be a separate port within the inspiratory gases flow path or may be integrated into the sealed patient interface.

The recirculation of exhaled gases helps to conserve oxygen within the pressure support respiratory apparatus.

According to a further aspect, the present disclosure relates to a respiratory support apparatus comprising
a flow generator configured to generate a flow of gases
an inspiratory circuit comprising one or more components,
a patient interface in fluid communication with a patient's airways,
an expiratory circuit comprising one or more components,
the inspiratory circuit extending between the patient interface and the flow generator,
the inspiratory circuit defining a gas flow passage from an outlet of the flow generator to the patient interface,
the expiratory circuit extending from the patient to the flow generator, the expiratory circuit defining a gas flow passage from the patient to an inlet of the flow generator,
   wherein the expiratory circuit configured to capture and recirculate some or all of the exhaled gases from a patient

The flow generator configured to generate suction within the expiratory circuit such that aerosols and other material from the exhaled gases are drawn away from the patient through the expiratory circuit.

The expiratory circuit comprises a expiratory conduit. The expiratory circuit further comprises a scrubber to scrub out CO₂ from the exhaled gases. The expiratory circuit may optionally comprise a disinfection unit.

The inspiratory circuit comprises an inspiratory conduit. The inspiratory circuit further comprises a humidifier downstream of the flow generator and configured to humidify the gases flow generated by the flow generator. The inspiratory conduit may optionally have a heater wire within it, wherein the heater wire is controlled by a controller of the flow generator or a controller of the humidifier or a single controller that is configured to control the humidifier and flow generator.

The respiratory support apparatus comprises a filter, wherein the filter is coupled to an inlet of the flow generator or the filter is positioned within the expiratory circuit prior to the inlet of the flow generator.

The respiratory support apparatus is a pressure therapy apparatus that is configured to deliver a pressure to a patient. The flow generator is controlled to deliver a set pressure. In the pressure therapy apparatus, the patient interface is a sealed patient interface, that seals with one or more airways of the patient. The pressure therapy apparatus further comprises a leak port positioned in the inspiratory circuit or on the patient interface. The expiratory circuit is in fluid communication with the leak port and configured to draw exhaled gases from the patient interface into the expiratory circuit via the leak port.

In the pressure therapy apparatus the expiratory circuit, inspiratory circuit and the patient interface form a closed gases path from the flow generator to the patient interface. In the pressure therapy apparatus, the flow generator may optionally comprise an oxygen inlet that is configured to introduce oxygen into the gases flow.

The respiratory support apparatus is a high flow therapy apparatus that is configured to deliver high flow to a patient. The flow delivered is humidified high flow and the flow generator is controlled to deliver a set flow. In the high flow therapy apparatus the patient interface is a first patient interface that is an unsealed interface e.g. a nasal cannula. The high flow therapy apparatus further comprises a second interface that does not seal with the patient's airways. The second patient interface is a face shield or cover that forms a shroud about a patient's airways and the nasal cannula. The second patient interface comprises one or more walls that are shaped or angled to direct exhaled gases and/or ambient air toward the expiratory circuit that is in fluid communication with the second patient interface.

In the high flow therapy apparatus the inspiratory circuit comprises a leak port upstream of the first patient interface and is configured to leak out a portion of the gases flow generated by the flow generator such that the suction created within the expiratory circuit causes a flow rate that is greater than the flow rate delivered to the first patient interface. The greater flow rate created by suction in the expiratory circuit may cause most or all of the exhaled gases to be drawn away from the patient and through the expiratory circuit.

The respiratory support apparatus as described is advantageous because it captures and draws exhaled gases away from the patient as opposed to venting or releasing the exhaled gases to ambient air. The apparatus cleans and recirculates the gases which is advantageous because it reduces the spread of aerosols or infectious diseases from patients due to the capture and recirculation of the exhaled gases. Further the capture and recirculation of exhaled gases is also advantageous because it allows conservation of oxygen as oxygen wasted to atmosphere (i.e. ambient) is reduced.

Further statements of aspects in accordance with this disclosure follow below. It will be appreciated that these following aspects can be combined with one, some, or any of the aspects of the disclosure in the statements above.

According to another aspect of this disclosure there is provided a respiratory support apparatus configured to provide a gases flow to a patient, the respiratory support apparatus comprising:
a flow generator configured to generate the gases flow;
a patient interface configured to be mounted on the patient and to receive the gases flow from the flow generator to deliver the gases flow to the patient;
wherein the apparatus comprises a leak port configured to provide a controlled leak of the gases flow; the leak port being configured to provide variable occlusion of the leak flow path through the leak port, the occlusion increasing as the pressure and/or flow rate of the gases flow increases.

The leak flow through the leak flow path may be substantially constant across a range of pressures and/or flow of the gases flow. The leak flow through the leak flow path may be substantially constant over the operating pressure/flow rate range of the flow generator. The leak port may comprise a plurality of leak apertures.

The leak port may comprise one or more leak valves.

The leak port may be in or at the flow generator. The respiratory support apparatus may comprise a flow generator housing, the leak port being located in the flow generator housing downstream of the flow generator and in fluid communication with the flow generator.

The respiratory support apparatus may comprise a breathing circuit configured to extend between, and be in fluid communication with the patient interface and the flow generator, to provide a flow path for the gases flow to the patient; the leak port being configured to provide a controlled leak of the gases flow from the breathing circuit.

The breathing circuit may comprise an inspiratory circuit between an outlet of the flow generator and the patient interface, for delivering breathing gases to the patient, the leak port being located in the inspiratory circuit.

The breathing circuit may comprise an expiratory circuit between an inlet of the flow generator and the patient interface, for receiving expiratory gases from the patient, the leak port being located in the expiratory circuit.

The leak port may comprise a valve, occlusion of the valve being actively controlled by a controller. The respiratory support apparatus may comprise the controller, the controller controlling the occlusion of the valve in response to a signal received by, or generated by, the controller, the signal being indicative of pressure and/or flow rate of the gases flow at the flow generator. The controller may be a controller of the flow generator, the controller controlling the pressure and/or flow rate of the gases flow generated by the flow generator.

The valve may be a passive valve, that is, a valve the occlusion of which varies without active control by a controller. In these embodiments, the valve automatically varies occlusion in response to pressure from the gases flow acting on the valve.

The passive valve may comprise a valve body configured to receive the leak flow, and a plunger movably mounted in the body, at least one of the body and the plunger comprising an aperture that can be exposed to the leak flow, movement of the plunger within the body varying occlusion of the aperture.

The respiratory support apparatus may comprise a plurality of leak apertures that can be exposed to the leak flow, movement of the plunger within the body varying occlusion of the apertures.

The plunger may comprise the aperture(s).

The plunger may be biased towards the aperture(s) being open. The plunger may be biased by a biasing member, preferably a spring.

The valve body may comprise a cylindrical valve body, the plunger and valve body being concentric.

The valve body and plunger may be telescopically mounted together.

The passive valve may comprise a valve body comprising a leak flow passage for the leak flow, and a pneumatic member in the valve body, pneumatically coupled to the gases flow, wherein varying the pressure and/or flow rate of the gases flow moves at least part of the pneumatic member to vary occlusion of the leak valve.

The pneumatic member may be substantially rigid, and is movably mounted on the valve body.

The pneumatic member may be resiliently deformable, so as to deform when subject to the pressure and/or flow rate of the gases flow.

The pneumatic member may be configured to change size and/or shape when subject to the pressure and/or flow rate of the gases flow, the change in size and/or shape varying occlusion of the leak port.

The pneumatic member may comprise a diaphragm, varying the pressure and/or flow rate of the gases flow moving at least part of the diaphragm within the body to vary occlusion of the leak valve.

The diaphragm may be configured to move substantially perpendicularly to the leak flow through the valve body.

The pneumatic member may comprise a bladder configured to be inflated by the gases flow, increasing the pressure and/or flow rate of the gases flow increasing the inflation of the bladder to increase occlusion of the leak valve.

The leak valve may comprise a pressure conduit in addition to the leak flow passage, the pressure conduit being in fluid communication with the gases flow, the pressure conduit feeding a proportion of the gases flow to the pneumatic member to move the pneumatic member.

The leak port and/or the amount of leak flow through the leak port, is controlled by the flow generator.

The respiratory support apparatus may comprise a plurality of leak ports.

The respiratory support apparatus may comprise a primary inlet configured to receive ambient air.

The respiratory apparatus may comprise a secondary inlet configured to receive a supplementary gas. The secondary inlet may be an oxygen inlet configured to receive oxygen from a supplementary oxygen supply.

According to another aspect of this disclosure there is provided a respiratory support apparatus configured to provide a gases flow to a patient, the respiratory support apparatus comprising:
a flow generator configured to generate the gases flow;
a patient interface configured to be mounted on the patient and to receive the gases flow from the flow generator to deliver the gases flow to the patient;
wherein the apparatus comprises a leak port or valve configured to provide a controlled leak of the gases flow; the leak port or valve being configured to provide variable occlusion of the leak flow path through the leak port or valve, such that the leak flow through the leak flow path is substantially constant across a range of pressures and/or flow rates of the gases flow.

The leak flow through the leak flow path may be substantially constant across a range of pressures and/or flow of the gases flow. The leak flow through the leak flow path may be substantially constant over the operating pressure/flow rate range of the flow generator.

According to another aspect of this disclosure there is provided a respiratory support apparatus configured to provide a gases flow to a patient, the respiratory support apparatus comprising:
a flow generator configured to generate the gases flow;
a patient interface configured to be mounted on the patient and to receive the gases flow from the flow generator to deliver the gases flow to the patient;
wherein the apparatus comprises a leak port configured to provide a controlled leak of the gases flow via a leak flow path; the leak port being configured such that the leak flow through the leak flow path does not substantially increase as the pressure and/or flow rate of the gases flow increases.

According to another aspect of this disclosure there is provided a respiratory support apparatus configured to provide a gases flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator to deliver the gases flow to the patient;
a breathing circuit comprising:
an inspiratory conduit configured to extend between, and be in fluid communication with, the patient interface and the flow generator outlet, to provide a flow path for inspiratory gases between the patient interface and the flow generator outlet;
an expiratory conduit configured to extend between, and be in fluid communication with, a region at or adjacent the patient interface and the flow generator inlet, to provide a flow path for expiratory gases between the region at or adjacent the patient interface and the flow generator inlet;
wherein the generation by the flow generator of the gases flow also generates suction forces in the expiratory conduit, such that the flow generator forces expiratory gases along the expiratory conduit from the region at or adjacent the patient interface; and
wherein the apparatus comprises a leak port configured to provide a controlled leak of the gases flow between the flow generator outlet and the patient interface; the leak port being configured:
to provide variable occlusion of the leak flow path through the leak port, the occlusion increasing as the pressure and/or flow rate of the gases flow through the breathing circuit increases; and/or
such that the leak flow through the leak flow path is substantially constant across a range of pressures and/or flow rates through the breathing circuit.

According to another aspect of this disclosure there is provided a respiratory support apparatus configured to provide a gases flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator to deliver the gases flow to the patient;
a breathing circuit comprising an expiratory conduit configured to extend between, and be in fluid communication with, a region at or adjacent the patient interface and the flow generator inlet, to provide a flow path for expiratory gases between the region at or adjacent the patient interface and the flow generator inlet;
wherein the generation by the flow generator of the gases flow also generates forces in the expiratory conduit, such that the flow generator scavenges expiratory gases from the region at or adjacent the patient interface; and
wherein the apparatus comprises a leak port configured to provide a controlled leak of the gases flow between the flow generator outlet and the patient interface; wherein the leak port is configured:

to provide variable occlusion of the leak flow path through the leak port, the occlusion increasing as the pressure and/or flow rate of the gases flow through the breathing circuit increases; and/or
such that the leak flow through the leak flow path is substantially constant across a range of pressures and/or flow rates through the breathing circuit.

According to another aspect of this disclosure there is provided a breathing circuit for a respiratory support apparatus, the breathing circuit comprising a leak port, the leak port configured to provide a controlled leak of the gases flow from the breathing circuit; wherein the leak port is configured:
to provide variable occlusion of the leak flow path through the leak port, the occlusion increasing as the pressure and/or flow rate of the gases flow through the breathing circuit increases; and/or
such that the leak flow through the leak flow path is substantially constant across a range of pressures and/or flow rates through the breathing circuit.

According to a further aspect of this disclosure there is provided a respiratory support apparatus configured to provide a gases flow to a patient, the respiratory support apparatus comprising:
a flow generator configured to generate the gases flow and comprising an inlet and an outlet, the flow generator being configured to draw inspiratory gases in through the inlet, and to deliver the gases flow through the outlet;
a patient interface configured to be mounted on the patient and to receive the gases flow from the flow generator outlet to deliver the gases flow to the patient;
an expiratory conduit configured to extend between, and be in fluid communication with, a region at or adjacent the patient interface and the flow generator inlet, to provide a flow path for expiratory gases between the region at or adjacent the patient interface and the flow generator inlet;
wherein the generation by the flow generator of the gases flow also forces expiratory gases along the expiratory conduit from the region at or adjacent the patient interface; and
wherein the apparatus comprises a leak port or valve configured to provide a controlled leak of the gases flow between the flow generator outlet and the patient interface;
   wherein the leak port is configured:
to provide variable occlusion of the leak flow path through the leak port, the occlusion increasing as the pressure and/or flow rate of the gases flow through the breathing circuit increases; and/or
such that the leak flow through the leak flow path is substantially constant across a range of pressures and/or flow rates through the breathing circuit.

According to a further aspect, the present disclosure related to a pressure support respiratory apparatus comprising:
a flow generator comprising an inlet and an outlet, the flow generator configured to generate a flow of gases, wherein the flow generator is pressure controlled,
an inspiratory gas flow path in fluid communication with the outlet of the flow generator,
the inspiratory gas flow path comprising an inspiratory conduit and a leak port, the inspiratory conduit and the leak port being fluidically coupled to each other,
a sealed patient interface in fluid communication with the inspiratory gas flow path, the sealed patient interface providing a flow of gases to the patient, wherein the sealed patient interface creating a pressure within a patient's airways,
an expiratory gas flow path in fluid communication with the leak port and the expiratory gas flow path in fluid communication with an inlet of the flow generator, wherein the expiratory gas flow path comprises an expiratory conduit,
wherein the flow generator creates a positive pressure within the inspiratory gas flow path to create a positive flow of gases toward the patient,
wherein a negative pressure or suction is created within the expiratory gas flow path when the flow generator is generating the flow of gases, and wherein the negative pressure draws exhaled gases out through the leak port,
wherein the inspiratory gas flow path, the patient interface and the expiratory gas flow path form a closed flow path (i.e. a sealed gas flow path) with minimal or no leak of gases;
wherein the apparatus comprises a leak port or valve configured to provide a controlled leak of the gases flow between the flow generator outlet and the patient interface;
wherein the leak port is configured:
to provide variable occlusion of the leak flow path through the leak port, the occlusion increasing as the pressure and/or flow rate of the gases flow through the breathing circuit increases; and/or
such that the leak flow through the leak flow path is substantially constant across a range of pressures and/or flow rates through the breathing circuit.

According to a further aspect, the present disclosure relates to a high flow respiratory support apparatus comprising:
a flow generator comprising an inlet and an outlet, the flow generator configured to generate a flow of gases, wherein the flow generator is flow controlled,
an inspiratory gas flow path in fluid communication with the outlet of the flow generator,
   the inspiratory gas flow path comprising an inspiratory conduit and a leak port, the inspiratory conduit and the leak port being fluidically coupled to each other,
a first patient interface in fluid communication with the inspiratory gas flow path, the first patient interface in fluid communication with a patient's nares and the first patient interface engaging the patient's nares in a non sealing manner,
an expiratory gas flow path in fluid communication with the inlet of the flow generator, the expiratory gas flow path comprising an expiratory conduit,
a second patient interface positioned on the patient, the second patient interface defining a cover that encompasses the first patient interface, wherein the second patient interface in fluid communication with the expiratory gas flow path,
wherein the flow generator creates a positive pressure within the inspiratory gas flow path to create a positive flow of gases toward the patient, a portion of the positive flow of gases is leaked out through the leak port such that the total flow or flow rate from the first interface to the patient is less than the flow rate or total flow generated by the flow generator,
wherein the a negative pressure or suction is created within the expiratory gas flow path when the flow generator is generating the flow of gases, and wherein the flow rate or total flow within the expiratory gas flow path is greater than the flow rate or total flow from the first patient interface, and;
wherein the first patient interface and second patient interface are both unsealed;
   wherein the apparatus comprises a leak port or valve configured to provide a controlled leak of the gases flow between the flow generator outlet and the patient interface;
   wherein the leak port is configured:
   to provide variable occlusion of the leak flow path through the leak port, the occlusion increasing as the pressure and/or flow rate of the gases flow through the breathing circuit increases; and/or
   such that the leak flow through the leak flow path is substantially constant across a range of pressures and/or flow rates through the breathing circuit.

According to a further aspect of this disclosure there is provided a respiratory pressure support apparatus configured to provide a gases flow to a patient, the apparatus comprising:
a delivery flow generator configured to generate the gases flow and comprising an inlet and an outlet, the delivery flow generator being configured to draw inspiratory gases in through the inlet, and to deliver the gases flow through the outlet;
a patient interface configured to be mounted on the patient and to receive the gases flow from the flow generator outlet;
a expiratory flow generator comprising a flow generator inlet;
an expiratory conduit configured to extend between, and be in fluid communication with, the patient interface and the flow generator inlet of the expiratory flow generator, to provide a flow path for expiratory gases between the patient interface and the flow generator inlet of the expiratory flow generator;
wherein the expiratory flow generator generates forces in the expiratory conduit, such that the expiratory flow generator forces expiratory gases along the expiratory conduit away from the patient interface.

The delivery and expiratory flow generators may be controlled such that the flow rate of the expiratory gases through the expiratory flow generator is greater than the flow rate of the gases flow through the delivery flow generator.

The flow rate of the expiratory gases through the expiratory flow generator may be greater than the flow rate of the gases flow through the delivery flow generator by a predetermined amount.

The predetermined amount may be controlled by the apparatus.

The predetermined amount can be varied by the apparatus.

The predetermined amount may be between 1 and 30 L/min, preferably 5 and 25 L/min, more preferably between 10 and 20 L/min, and most preferably between 13 and 17 L/min, in some embodiments may be 15 L/min, and in some embodiments may be greater than 15 L/min.

The apparatus may comprise a controller configured to control the delivery flow generator and to control the expiratory flow generator.

The apparatus may comprise a delivery controller configured to control the delivery flow generator, and an expiratory controller to control the expiratory flow generator.

A communication link may be provided between the delivery and expiratory controllers.

The communication link may be a wired or a wireless link.

The communication link may be configured such that one controller comprises a master controller, and the other controller comprises a slave controller, the communication link being configured to permit the master controller to control the slave controller.

The master controller may be configured to make a first determination, or to receive an input being a first determination, of the flow rate of the gases flow through the delivery flow generator or the flow rate of the expiratory gases flow through the expiratory flow generator.

The master controller may subsequently make a second determination of the flow rate of the other of the flow rate of the gases flow through the delivery flow generator or the flow rate of the expiratory gases flow through the expiratory flow generator, the second determination being made in dependence upon the first determination.

The controller, or one of the controllers, may be configured to activate one flow generator before the other flow generator.

The expiratory flow generator may be activated before the delivery flow generator.

The controller, or one of the controllers, may be configured to generate a signal indicative of whether or not the flow rate of expiratory gases through the expiratory flow generator is greater than the flow rate of the gases flow through the delivery flow generator and to generate a signal.

If the signal is indicative that the flow rate of expiratory gases through the expiratory flow generator is not greater than the flow rate of the gases flow through the delivery flow generator, the controller, or one of the controllers, may be configured to:
a. increase the speed of rotation of an impeller of the expiratory flow generator; and/or
b. decrease the speed of rotation of an impeller of the delivery flow generator.

The apparatus may comprise a second patient interface configured to cover at least the nose and mouth of the patient; the expiratory conduit being configured to extend between the second patient interface and the flow generator inlet of the expiratory flow generator.

The apparatus may comprise a wireless communications module configured to receive one or more control signals from a remote peripheral device, such that peripheral device can control the delivery flow generator and/or the expiratory flow generator.

The apparatus may comprise an apparatus housing, the delivery and expiratory flow generators being provided in the apparatus housing.

The apparatus may comprise two flow generator apparatus: a delivery flow generator apparatus comprising the delivery flow generator, and a expiratory flow generator apparatus comprising the expiratory flow generator.

Each apparatus may comprise its own apparatus housing.

Each flow generator may be separate from the other, the flow generator apparatus being connected only via a communications link extending between the flow generator apparatus.

The delivery flow generator apparatus and the expiratory flow generator apparatus may be substantially the same.

The delivery flow generator apparatus and the expiratory flow generator apparatus may be interchangeable, such that either apparatus can be configured to provide the gases flow or provide removal of the expiratory gases flow.

The delivery flow generator apparatus may comprise any one or more of the following:
a. a humidifier configured to humidify the gases flow;
b. a filter configured to filter the gases flow;
c. a supplementary gases inlet, such as an oxygen inlet.

The expiratory flow generator apparatus may comprise any one or more of the following:
a. a filter configured to filter the expiratory gases flow;
b. an ambient outlet port or valve, configured to allow the expiratory gases flow to outlet the flow generator to ambient.

According to another aspect of this disclosure there is provided a respiratory pressure support apparatus configured to provide a gases flow to a patient, the apparatus comprising:
a. a delivery flow generator configured to generate the gases flow, and comprising an inlet and an outlet, the delivery flow generator being configured to draw inspiratory gases in through the inlet, and to deliver the gases flow through the outlet;
b. a patient interface configured to be mounted on the patient and to receive the gases flow from the flow generator outlet;
c. an expiratory flow generator comprising a flow generator inlet;
d. an expiratory conduit configured to extend between, and be in fluid communication with, the patient interface and the flow generator inlet of the expiratory flow generator, to provide a flow path for expiratory gases between the patient interface and the flow generator inlet of the expiratory flow generator;
wherein the expiratory flow generator generates forces in the expiratory conduit, such that the expiratory flow generator forces expiratory gases along the expiratory conduit from the patient interface;
wherein the first and expiratory flow generators are controlled such that the flow rate of the expiratory gases through the expiratory flow generator is greater than the flow rate of the gases flow through the delivery flow generator.

According to a further aspect of this disclosure there is provided a respiratory pressure support apparatus configured to provide a gases flow to a patient, the apparatus comprising:
a. a delivery flow generator configured to generate the gases flow, and comprising an inlet and an outlet, the delivery flow generator being configured to draw inspiratory gases in through the inlet, and to deliver the gases flow through the outlet;
b. a patient interface configured to be mounted on the patient and to receive the gases flow from the flow generator outlet;
c. a second patient interface configured to cover at least the nose and mouth of the patient;
d. a expiratory flow generator comprising a flow generator inlet;
e. an expiratory conduit configured to extend between, and be in fluid communication with, the second patient interface and the flow generator inlet of the expiratory flow generator, to provide a flow path for expiratory gases between the second patient interface and the flow generator inlet of the expiratory flow generator;
wherein the expiratory flow generator generates forces in the expiratory conduit, such that the expiratory flow generator forces expiratory gases along the expiratory conduit from inside the second patient interface.

The apparatus may comprise an expiratory gases outlet port or valve through which expiratory gases can flow to ambient, from the expiratory flow generator.

The apparatus may comprise an expiratory gases filter configured to filter aerosols and/or pathogens from the expiratory gases.

The filter may comprise any one or more of:
a. a HEPA filter;
b. a pleated filter;
c. a virus filter, such as a PM1 filter;
d. a layered filter, each layer configured to filter a different component from the expiratory gases.

The filter may be upstream of the flow generator inlet of the expiratory flow generator.

The expiratory flow generator may be provided in a housing, the filter being internal of the housing.

The expiratory flow generator may be provided in a housing, the filter being external of the housing.

The filter may be provided in the expiratory conduit.

The filter may be downstream of the expiratory flow generator.

The filter may be provided in a filter cartridge, the filter cartridge being removably mounted on the apparatus.

The apparatus may comprise multiple filters.

The apparatus may be configured to provide high flow therapy, such as nasal high flow therapy.

Features from one or more embodiments or configurations may be combined with features of one or more other embodiments or configurations. Additionally, more than one embodiment may be used together during a process of respiratory support of a patient.

The term ‘comprising’ as used in this specification means ‘consisting at least in part of’. When interpreting each statement in this specification that includes the term ‘comprising’, features other than that or those prefaced by the term may also be present. Related terms such as ‘comprise’ and ‘comprises’ are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

It should be understood that alternative embodiments or configurations may comprise any or all combinations of two or more of the parts, elements or features illustrated, described or referred to in this specification.

This invention may also be said broadly to consist in the parts, elements and features referred to or indicated in the specification of the application, individually or collectively, and any or all combinations of any two or more said parts, elements or features.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth.

### BRIEF DESCRIPTION OF THE DRAWINGS

FIG. 1A shows in diagrammatic form a respiratory support apparatus being a high flow respiratory support apparatus.

FIG. 1B illustrates a sensing circuit board including a flow rate sensor that may be used in a respiratory support apparatus.

FIG. 1C shows a schematic view of a known respiratory pressure support apparatus being a CPAP/Bilevel apparatus, in which the blower unit and humidifier unit are integrated into a single main housing.

FIG. 1D shows a schematic view of the main components and configuration of a respiratory pressure support apparatus being a CPAP/Bilevel apparatus with a humidifier.

FIG. 2 is a schematic view of a first embodiment of a high flow respiratory support apparatus in accordance with this disclosure.

FIG. 3 is a schematic view of a second embodiment of a high flow respiratory support apparatus in accordance with this disclosure.

FIG. 4 is a schematic view of a third embodiment of a high flow respiratory support apparatus in accordance with this disclosure.

FIG. 5 is a schematic gas flow path diagram of a filter module and a valve module that could comprise part of a respiratory support apparatus in accordance with this disclosure, with the solid line arrows representing the flow of oxygen (or another gas), and the dashed line arrows representing the flow of ambient air.

FIGS. 6A and 6B are views of a leak port in accordance with this disclosure.

FIG. 7 is a schematic view of an embodiment of a respiratory pressure support apparatus being a CPAP or Bilevel apparatus in this example.

FIG. 8 is a schematic view of a third embodiment of a high flow respiratory support apparatus in accordance with other aspects of this disclosure.

FIG. 9 is a schematic view of a third embodiment of a high flow respiratory support apparatus in accordance with other aspects of this disclosure.

FIG. 10 is a schematic enlarged view of part of a high flow respiratory support apparatus in accordance with this disclosure, showing an example of a leak valve.

FIG. 11 is a schematic enlarged view of part of a high flow respiratory support apparatus in accordance with this disclosure, showing another example of a leak valve.

FIG. 12 is a schematic view of a fourth embodiment of a high flow respiratory support apparatus in accordance with other aspects of this disclosure, showing a mount comprising a strap for securing one end of the expiratory conduit near the patient.

FIG. 13 is a schematic view of the high flow respiratory support apparatus of FIG. 12 showing a different mount comprising a collar for securing one end of the expiratory conduit near the patient.

FIG. 14 is a schematic view of an apparatus in accordance with other aspects of this disclosure, including a face shield in the form of a hood over the patient's head.

FIG. 15 is a schematic view of a sixth embodiment of a high flow respiratory support apparatus in accordance with other aspects of this disclosure, comprising a leak flow conduit.

FIG. 16 is a schematic view of a seventh embodiment of a high flow respiratory support apparatus in accordance with other aspects of this disclosure, comprising one or more leak flow ports at the patient interface.

FIG. 17 is a schematic view of an eighth embodiment of a high flow respiratory support apparatus in accordance with other aspects of this disclosure, comprising an oxygen concentrator and a supplementary oxygen supply.

FIG. 18 is a schematic view of a ninth embodiment of a high flow respiratory support apparatus in accordance with other aspects of this disclosure, comprising an oxygen concentrator without a supplementary oxygen supply.

FIG. 19 is a schematic view of a tenth embodiment of a high flow respiratory support apparatus in accordance with other aspects of this disclosure, comprising a gases delivery apparatus and a gases expiratory apparatus, and a communication link between them.

FIG. 20 is a schematic view of the apparatus of FIG. 19, also comprising a wireless communication link between the apparatus and a mobile peripheral device.

FIG. 21 is a flow diagram showing set-up and start-up steps of the apparatus of FIG. 19.

FIG. 22 is a flow diagram showing set-up and start-up steps of the apparatus of FIG. 20, using the mobile peripheral device.

FIG. 23 is a flow diagram showing control steps used to adjust gases flow rates of the apparatus of FIG. 19.

FIG. 24 is a flow diagram showing control steps used to adjust gases flow rates of the apparatus of FIG. 20, using the mobile peripheral device.

### DETAILED DESCRIPTION

It is known in the art to provide a CPAP/Bi-Level respiratory support apparatus comprising a flow generator in the form of a blower which draws ambient air into an impeller to generate an inspiratory gases flow which is delivered to the patient via one or more inspiratory conduits and a patient interface mounted on the head of the patient. Such apparatus often comprises a humidifier, downstream of the blower, to humidify the inspiratory gases flow prior to delivery to the patient. In some examples, the inspiratory conduit can be heated. In some examples, the patient interface comprises a full face mask configured to seal around the nose and mouth of the patient. Typically, such masks include an exhalation port or valve to allow exhaled gases to exit the mask as the patient breaths. Such a respiratory support apparatus can be controlled for example to a user (e.g. clinician) set pressure of 10cmH₂O, generating an example inspiratory gases flow. In such a pressure controlled apparatus, the inspiratory gases flow is controlled to a user set pressure. The flow rate of the inspiratory gases flow varies according to the pressure set. For example, if the set pressure is 10cmH₂O, an example inspiratory gases flow might be generated of around 40 L/min. When controlled to a set pressure of 20cmH₂O, an example inspiratory gases flow might be generated at a flow rate of around 55 L/min.

It is also known to provide a High Flow respiratory support apparatus. One example of such a high flow respiratory support apparatus comprises a patient interface being a nasal cannula comprising prongs received in the nares of the patient. In such an apparatus the patient typically exhales directly to atmosphere via leak around the prongs of the cannula. In some cases, there can also be some leak flow via the patient's mouth. In such an example such a respiratory support apparatus can be controlled to a user defined flow rate, generating an example inspiratory gases flow at a flow rate of around 50-60/min. In an alternative form a high flow respiratory support apparatus may comprise a trachea patient interface to deliver high flow through a tracheal tube inserted into the neck of a patient.

In the above examples, the pressures and flow rates are examples only, to illustrate the type of respiratory support (i.e. respiratory therapy) that such apparatus can deliver to a patient. Further, more detailed, examples are provided below.

In accordance with aspects of this disclosure we provide a respiratory support apparatus capable of providing pressure therapy, or high flow therapy. The apparatus may be configured to be operative according to a different mode associated with each type of therapy. The mode, and therefore the therapy, may be selected by the user.

### Respiratory Support Apparatus

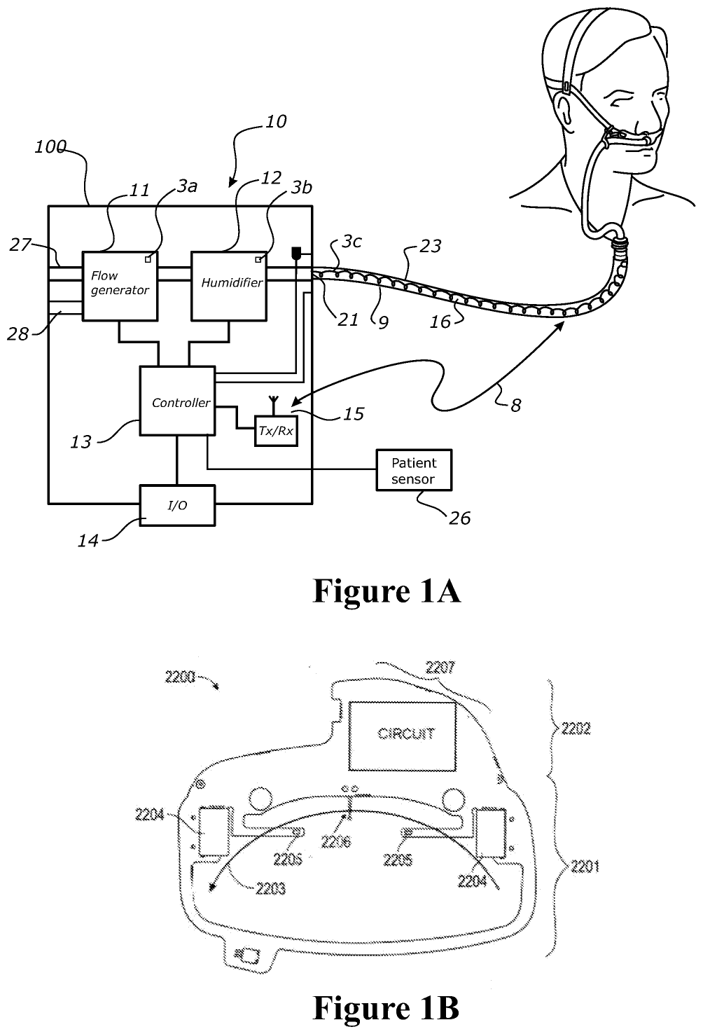

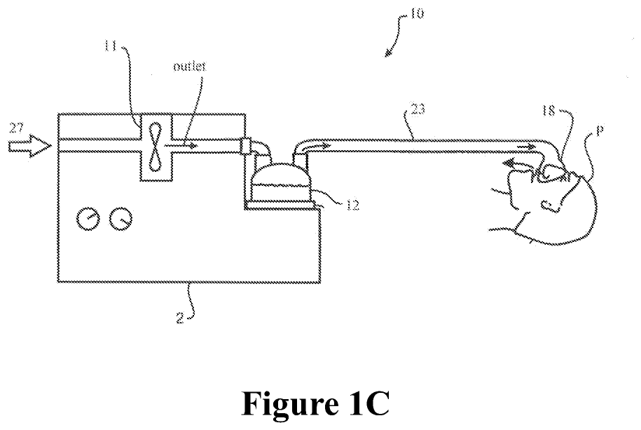

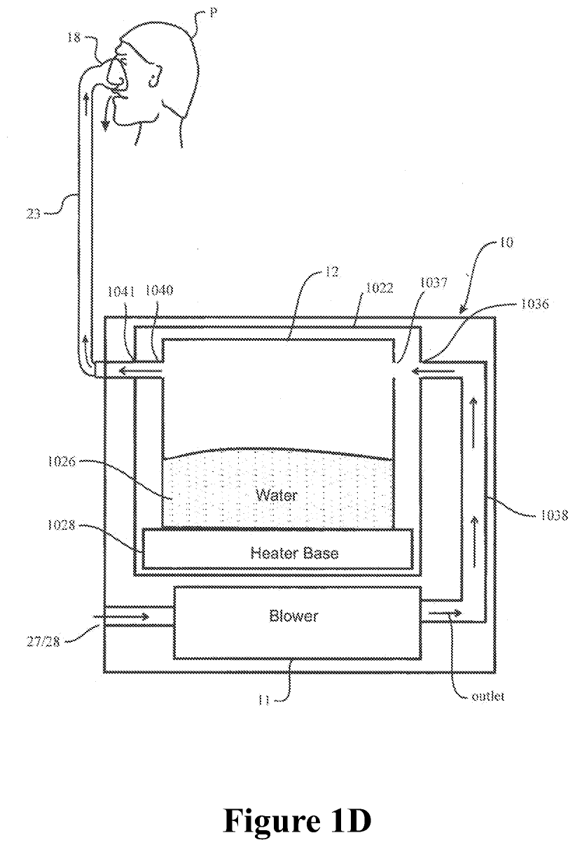

A respiratory support apparatus 10 is shown in FIG. 1A. The respiratory support apparatus 10 can comprise a main housing 100 that contains a flow generator 11 in the form of a motor/impeller arrangement (for example, a blower), an optional humidifier 12, a controller 13, and a user interface 14 (comprising, for example, a display and input device(s) such as button(s), a touch screen, or the like). The controller 13 can be configured or programmed to control the operation of the apparatus. For example, the controller can control components of the apparatus, including but not limited to: operating the flow generator 11 to create a flow of gas (gases flow) for delivery to a patient, operating the humidifier 12 (if present) to humidify and/or heat the generated gases flow, control a flow of oxygen into the flow generator blower, receiving user input from the user interface 14 for reconfiguration and/or user-defined operation of the apparatus 10, and outputting information (for example on the display) to the user. The user can be a patient, healthcare professional, or anyone else interested in using the apparatus. As used herein, a “gases flow” can refer to any flow of gases that may be used in the breathing assistance or respiratory device, such as a flow of ambient air, a flow comprising substantially 100% oxygen, a flow comprising some combination of ambient air and oxygen, and/or the like.

A patient breathing conduit 16 is coupled at one end to a gases flow outlet 21 in the housing 100 of the respiratory support apparatus 10. The patient breathing conduit 16 is coupled at another end to a patient interface 17 such as a non-sealed nasal cannula with a manifold 19 and nasal prongs 18. Additionally, or alternatively, the patient breathing conduit 16 can be coupled to a face mask, a nasal mask, a nasal pillows mask, an endotracheal tube, a tracheostomy interface, and/or the like. Preferably in the respiratory support apparatus 10 the patient interface 17 is an unsealed interface in order to provide high flow respiratory support to a patient. The gases flow that is generated by the respiratory support apparatus 10 may be humidified, and delivered to the patient via the patient conduit 16 through the cannula 17. The patient conduit 16 can have a heater wire 16a to heat gases flow passing through to the patient. The heater wire 16a can be under the control of the controller 13. The patient conduit 16 and/or patient interface 17 can be considered part of the respiratory support apparatus 10, or alternatively peripheral to it. The respiratory support apparatus 10, breathing conduit 16, and patient interface 17 together can form a respiratory support system.

The controller 13 can control the flow generator 11 to generate a gases flow of the desired flow rate. The controller 13 can also control a supplemental oxygen inlet to allow for delivery of supplemental oxygen, the humidifier 12 (if present) can humidify the gases flow and/or heat the gases flow to an appropriate level, and/or the like. The gases flow is directed out through the patient conduit 16 and cannula 17 to the patient. The controller 13 can also control a heating element in the humidifier 12 and/or the heating element 16a in the patient conduit 16 to heat the gas to a desired temperature for a desired level of therapy and/or level of comfort for the patient. The controller 13 can be programmed with or can determine a suitable target temperature of the gases flow.

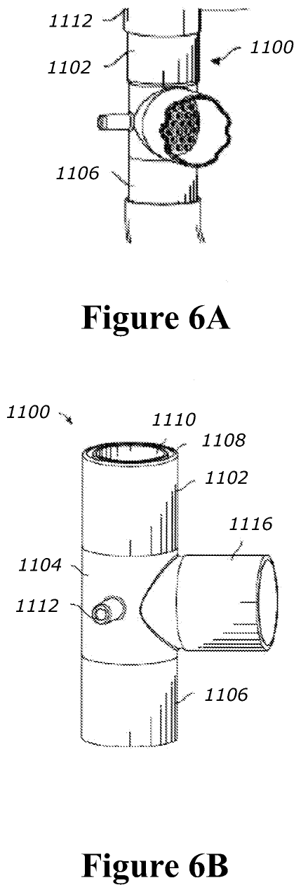

The oxygen inlet port 28 can include a valve through which a pressurized gas may enter the flow generator or blower. The valve can control a flow of oxygen into the flow generator blower. The valve can be any type of valve, including a proportional valve or a binary valve. The source of oxygen can be an oxygen tank or a hospital oxygen supply. Medical grade oxygen is typically between 95% and 100% purity. Oxygen sources of lower purity can also be used. Examples of valve modules and filters are disclosed in U.S. Provisional Application No. 62/409,543, titled “Valve Modules and Filter”, filed on Oct. 18, 2016, and U.S. Provisional Application No. 62/488,841, titled “Valve Modules and Filter”, filed on Apr. 23, 2017, which are hereby incorporated by reference in their entireties. Valve modules and filters are discussed in further detail below with relation to FIG. 6.

The respiratory support apparatus 10 can, in some examples, measure and control the oxygen content of the gas being delivered to the patient, and therefore the oxygen content of the gas inspired by the patient. During high flow respiratory support or treatment (i.e. high flow therapy), the high flow rate of gas delivered meets or exceeds the peak inspiratory demand of the patient. This means that the volume of gas delivered by the device to the patient during inspiration meets, or is in excess of, the volume of gas inspired by the patient during inspiration. High flow respiratory support (i.e. high flow therapy) therefore helps to prevent entrainment of ambient air when the patient breathes in, as well as flushing the patient's airways of expired gas. So long as the flow rate of delivered gas meets or exceeds peak inspiratory demand of the patient, entrainment of ambient air is prevented, and the gas delivered by the device is substantially the same as the gas the patient breathes in. As such, the oxygen concentration measured in the device, fraction of delivered oxygen, (FdO2) would be substantially the same as the oxygen concentration the user is breathing, fraction of inspired oxygen (FiO2), and as such the terms may can be seen as equivalent. Note that in other examples, apparatus 10 may not include any oxygen control, and may provide high flow to a patient without any supplementary oxygen being provided at all, i.e. the inspiratory gases flow to the patient is the flow generated from ambient air through the flow generator inlet, which may be optionally humidified.

Operation sensors 3a, 3b, 3c, such as flow, temperature, humidity, and/or pressure sensors can be placed in various locations in the respiratory support apparatus 10. Additional sensors (for example, sensors 20, 25) may be placed in various locations on the patient conduit 16 and/or cannula 17 (for example, there may be a temperature sensor 29 at or near the end of the inspiratory tube). Output from the sensors can be received by the controller 13, to assist the controller in operating the respiratory support apparatus 10 in a manner that provides suitable therapy. In some configurations, providing suitable therapy includes meeting a patient's peak inspiratory demand. The apparatus 10 may have a transmitter and/or receiver 15 to enable the controller 13 to receive signals 8 from the sensors and/or to control the various components of the respiratory support apparatus 10, including but not limited to the flow generator 11, humidifier 12, and heater wire 16a, or accessories or peripherals associated with the respiratory support apparatus 10. Additionally, or alternatively, the transmitter and/or receiver 15 may deliver data to a remote server or enable remote control of the apparatus 10.

Oxygen may be measured by placing one or more gas composition sensors (such as an ultrasonic transducer system, also referred to as an ultrasonic sensor system) after the oxygen and ambient air have finished mixing. The measurement can be taken within the device, the delivery conduit, the patient interface, or at any other suitable location. The particular sensor or sensors used can be varied depending on control requirements. In one example the ultrasonic transducer system is positioned within the gases flow path, within the housing of the respiratory support apparatus.

The respiratory support apparatus 10 may, in some examples, include a patient sensor 26, such as a pulse oximeter or a patient monitoring system, to measure one or more physiological parameters of the patient, such as a patient's blood oxygen saturation (SpO2), heart rate, respiratory rate, perfusion index, and provide a measure of signal quality. The sensor 26 can communicate with the controller 13 through a wired connection or by communication through a wireless transmitter on the sensor 26. The sensor 26 may be a disposable adhesive sensor designed to be connected to a patient's finger. The sensor 26 may be a non-disposable sensor. Sensors are available that are designed for different age groups and to be connected to different locations on the patient, which can be used with the respiratory support apparatus. The pulse oximeter would be attached to the user, typically at their finger, although other places such as an earlobe are also an option. The pulse oximeter would be connected to a processor in the device and would constantly provide signals indicative of the patient's blood oxygen saturation. The patient sensor 26 can be a hot swappable device, which can be attached or interchanged during operation of the respiratory support apparatus 10. For example, the patient sensor 26 may connect to the respiratory support apparatus 10 using a USB interface or using wireless communication protocols (such as, for example, near field communication, WiFi or Bluetooth®). When the patient sensor 26 is disconnected during operation, the respiratory support apparatus 10 may continue to operate in its previous state of operation for a defined time period. After the defined time period, the respiratory support apparatus 10 may trigger an alarm, transition from automatic mode to manual mode, and/or exit control mode (e.g., automatic mode or manual mode) entirely. The patient sensor 26 may be a bedside monitoring system or other patient monitoring system that communicates with the respiratory support apparatus 10 through a physical or wireless interface.

The respiratory support apparatus 10 may comprise a high flow respiratory support apparatus. High flow respiratory support as discussed herein is intended to be given its typical ordinary meaning as understood by a person of skill in the art, which generally refers to a respiratory assistance system delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen liters per minute (LPM) to about seventy liters per minute or greater. Typical flow rates for pediatric patients (such as neonates, infants and children) often range from, but are not limited to, about one liter per minute per kilogram of patient weight to about three liters per minute per kilogram of patient weight or greater. High flow respiratory support (i.e. high flow therapy) can also optionally include gas mixture compositions including supplemental oxygen and/or administration of therapeutic medicaments. High flow respiratory support is often referred to as nasal high flow (NHF), humidified high flow nasal cannula (HHFNC), high flow nasal oxygen (HFNO), high flow therapy (HFT), or tracheal high flow (THF), among other common names. The flow rates used to achieve “high flow” (i.e. high flow respiratory support) may be any of the flow rates listed below. For example, in some configurations, for an adult patient ‘high flow respiratory support’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between 25 LPM and 75 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow respiratory support’ (i.e. high flow therapy) may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow respiratory support apparatus with an adult patient, a neonatal, infant, or child patient, may deliver gases to the patient at a flow rate of between about 1 LPM and about 100 LPM, or at a flow rate in any of the sub-ranges outlined above. The respiratory support apparatus 10 can deliver any concentration of oxygen (e.g., FdO2), up to 100%, at any flowrate between about 1 LPM and about 100 LPM. In some configurations, any of the flowrates can be in combination with oxygen concentrations (FdO2s) of about 20%-30%, 21%-30%, 21%-40%, 30%-40%, 40%-50%, 50%-60%, 60%-70%, 70%-80%, 80%-90%, and 90%-100%. In some combinations, the flow rate can be between about 25 LPM and 75 LPM in combination with an oxygen concentration (FdO2) of about 20%-30%, 21%-30%, 21%-40%, 30%-40%, 40%-50%, 50%-60%, 60%-70%, 70%-80%, 80%-90%, and 90%-100%. In some configurations, the respiratory support apparatus 10 may include safety thresholds when operating in manual mode that prevent a user from delivering to much oxygen to the patient.

High flow respiratory support may be administered to the nares of a user and/or orally, or via a tracheostomy interface. High flow respiratory support may deliver gases to a user at a flow rate at or exceeding the intended user's peak inspiratory flow requirements. The high flow respiratory support may generate a flushing effect in the nasopharynx such that the anatomical dead space of the upper airways is flushed by the high incoming gases flow. This can create a reservoir of fresh gas available for each and every breath, while minimizing re-breathing of nitrogen and carbon dioxide. Meeting inspiratory demand and flushing the airways is additionally important when trying to control the patient's FdO2. High flow respiratory support can be delivered with a non-sealing patient interface such as, for example, a nasal cannula. The nasal cannula may be configured to deliver breathing gases to the nares of a user at a flow rate exceeding the intended user's peak inspiratory flow requirements.

The term “non-sealing patient interface” as used herein can refer to an interface providing a pneumatic link between an airway of a patient and a gases flow source (such as from flow generator 11) that does not completely occlude the airway of the patient. Non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or more of a nare or mouth of the patient. For a nasal cannula the airway is through the nares.

The flow generator or blower 11 can include an ambient air inlet port 27 to entrain ambient room air into the blower. The respiratory support apparatus 10 may also include an oxygen inlet port 28 leading to a valve through which a pressurized gas may enter the flow generator or blower 11. The valve can control a flow of oxygen into the flow generator blower 11. The valve can be any type of valve, including a proportional valve or a binary valve.

The blower can operate at a motor speed of greater than about 1,000 RPM and less than about 30,000 RPM, greater than about 2,000 RPM and less than about 21,000 RPM, greater than about 4,000 RPM and less than about 19,000 RPM or between any of the foregoing values. Operation of the blower can mix the gases entering the blower through the inlet ports. Using the blower as the mixer can decrease the pressure drop that would otherwise occur in a system with a separate mixer, such as a static mixer comprising baffles, because mixing requires energy. Having a static mixer can also increase the volume of the gas flow path between the valve and the gases composition sensor, which can further increase the delay between when the valve current is changed and when a corresponding change in oxygen concentration is measured.

Based on user inputs and the therapy supplied by the specific device, the controller can determine a target output parameter for the blower. The controller can receive measurements of the target output parameter, and based on the difference between determined flow rate and the measured flow rate, the controller can adjust the speed of the blower.

The target output parameter may be flow rate. The target flow rate may be a constant value, (e.g., nasal high flow). The target flow rate may be a value that fluctuates. In some configurations, the controller can control a blower motor speed based on a target flow rate, and additionally increase or decrease the motor speed based on a patient's breathing cycle. The target flow rate does not necessarily change, but the controller causes the motor speed to fluctuate in order add oscillations to the instantaneous flow rate such that the flow rate is synchronized with the patient's breathing. Such a system is described in International Application No. PCT/NZ2017/050063, titled “Flow Path Sensing for Flow Therapy Apparatus”, filed on May 17, 2017.

The target output parameter may alternatively be pressure. The target pressure may be a constant value, (e.g., CPAP). Alternatively, the target flow rate may a value that fluctuates, potentially in time with the breath, (e.g., bi-level NIV). In both of these scenarios, the total flow rate is unlikely to be constant. When controlling to pressure, the patient interface is changed by the user to a sealing interface that seals with the nose, or nose and mouth, of the patient to generate pressure.

With additional reference to FIG. 1B, a sensing circuit board 2200 is shown that can be implemented in the respiratory support apparatus 10. The sensing circuit board 2200 can be positioned in a sensor chamber such that the sensing circuit board 2200 is at least partially immersed in the flow of gases. The flow of gases may exit the blower 11 through a conduit and enter a flow path in the sensor chamber. At least some of the sensors on the sensing circuit board 2200 can be positioned within the flow of gases to measure gas properties within the flow. After passing through the flow path in the sensor chamber, the gases can exit to the humidifier 12 described above.

The sensing circuit board 2200 can be a printed sensing circuit board (PCB). Alternatively, the circuit on the board 2200 can be built with electrical wires connecting the electronic components instead of being printed on a circuit board. At least a portion of the sensing circuit board 2200 can be mounted outside of a flow of gases. The flow of gases can be generated by the flow generator 11 described above. The sensing circuit board 2200 can comprise ultrasonic transducers 2204. The sensing circuit board 2200 can comprise one or more of thermistors 2205. The thermistors 2205 can be configured to measure a temperature of the gases flow. The sensing circuit board 2200 can comprise a thermistor flow rate sensor 2206. The sensing circuit board 2200 can comprise other types of sensors, such as humidity sensors including humidity only sensors to be used with a separate temperature sensor and combined humidity and temperature sensors, sensors for measuring barometric pressure, sensors for measuring differential pressure, and/or sensors for measuring gauge pressure. The thermistor flow rate sensor 2206 can comprise hot wire anemometer, such as a platinum wire, and/or a thermistor, such as a negative temperature coefficient (NTC) or positive temperature coefficient (PTC) thermistor. Other non-limiting examples of the heated temperature sensing element include glass or epoxy-encapsulated or non-encapsulated thermistors. The thermistor flow rate sensor 2206 can be configured to measure flow rate of the gases by being supplied with a constant power, or be maintained at a constant sensor temperature or a constant temperature difference between the sensor and the flow of gases.

The sensing circuit board 2200 can comprise a first portion 2201 and a second portion 2202. The first portion 2201 can be positioned to be within the flow path of the gases, whereas the second portion 2202 can be positioned to be outside the flow path of the gases. The direction of the flow of gases is indicated in FIG. 1B by the arrow 2203. The direction of the flow of gases can be a straight line, or curved in shown in FIG. 1B.

Positioning the one or more of thermistors 2205 and/or the thermistor flow rate sensor 2206 downstream of the combined blower and mixer can take into account heat supplied to the gases flow from the blower. Also, immersing the temperature-based flow rate sensors in the flow path can increase the accuracy of measurements because the sensors being immersed in the flow can more likely to be subject to the same conditions, such as temperature, as the gases flow and therefore provide a better representation of the gases characteristics.

The sensing circuit board 2200 can comprise ultrasonic transducers, transceivers, or sensors of the sensing circuit board to measure gases properties of the gases flow, such as gas composition or concentration of one or more gases within the gases stream. Any suitable transducer, transceiver, or sensor may be mounted to the sensing circuit board 2200 as will be appreciated. For example, the sensing circuit board can include an ultrasonic transducer system (also referred to as an ultrasonic sensor system) that employs ultrasonic or acoustic waves for determining gas concentrations.

Positioning sensors in the flow path or module for example, instead of outside the flow path or module, allows the transducers 2204 to both operate within a smaller temperature range relative to one another, or both substantially at one temperature (namely, the temperature of the gas flow). Having them at a substantially homogenous temperature increases accuracy as the transducers are sensitive to temperature. Further, positioning sensors along the flow path allows for measurements and calculations that account for the influence of the gas velocity so that the effect of gas velocity can be removed from the sensor measurement.

In some configurations, the respiratory support apparatus may also be provided with a humidity sensor that is located in the flow path and which is configured to generate a humidity signal indicative of the humidity of the gases stream flowing through the sensor assembly. In such embodiments, the gas composition may be determined by the sensed speed of sound, and the sensed temperature and/or sensed humidity. The humidity sensor may be a relative humidity sensor or an absolute humidity sensor. In some embodiments, the gas composition may be determined based on the sensed speed of sound and the sensed humidity, without the need for a temperature sensor.

Pressure Support Apparatus, for example, CPAP/BiLevel Apparatus

FIG. 1C shows a schematic view of one type of known respiratory support apparatus 10 for delivering humidified and heated gases to a patient. The apparatus 10 comprises a housing containing a flow generator in the form of a blower unit 11, and a humidifier unit 12. In operation, atmospheric air 5 is drawn into the blower unit 11. The blower unit 11 generates a pressurized air or gases stream which is delivered to the inlet of a humidification chamber of the humidifier unit 12. The humidification chamber comprises water and is heated by a heater pad or plate. The humidified and heated gases stream exits the humidification chamber via an outlet of the humidification chamber and is delivered to the patient or user P via a flexible hose or gases conduit 23 and user interface 18 as shown. The blower unit 11 and humidification unit 12 are typically connected via a series of connectors and/or conduits to allow gases to pass from the blower unit 11 to the humidifier unit 12.

The user interface 18 shown in FIG. 1C is a nasal mask, covering the nose of the user P. However, it should be noted that in apparatus of these types, a mask that covers the mouth and nose, a full face mask, a nasal interface with sealing prongs (such as nasal pillows), or any other suitable user interface could be substituted for the nasal mask shown. A mouth-only interface or oral mask could also be used. The patient interface 18, in a pressurised support apparatus, is a sealed interface that forms a seal with the airways of the patient P.

The humidification chamber typically comprises a rigid plastic receptacle or container that can be filled with a volume of water. The humidification chamber may comprise a plastic material, such as a rigid plastic material. In one known form, the base of the humidification chamber comprises heater plate. An example heater plate is a circular, thermally conductive metal heater plate that is fixed on the base of the humidification chamber. The heater plate may be fixed within a complementary aperture provided in the base of the humidification chamber via, for example, overmolding of the plastic base about the peripheral edge of the heater plate. The overmolding forms a seal at the interface between the perimeter edge of the heater plate and surrounding plastic base surface of the chamber. In use, the heater plate contacts a heater pad or heater base upon which the humidification chamber rests and heats the volume of water in the chamber via conduction.

FIG. 1D shows an example of a typical schematic configuration of a respiratory pressure support apparatus 10 with a humidification chamber. The pressure support apparatus for example is a CPAP or BiLevel apparatus. The respiratory support apparatus 10 in this example comprises a humidification unit or humidification compartment 1022 that receives and retains a removable humidification chamber 1024 in use. In this embodiment, the humidification compartment 1022 may be formed within the housing of the respiratory apparatus 10 and may be an openable compartment having a lid so the humidification chamber 1024 within the humidification compartment 1022 may be accessed for removal for cleaning or filling. Typically, the humidification compartment 1022 is sealed and/or pressurised when the lid is closed. The humidification chamber 1024 is filled with a volume of water as indicated at 1026 and the chamber 1024 rests upon a heater pad or heater base 1028. As is known in the art, the heater pad 1028 is powered to heat the water 1026 in the humidification chamber 1024 during use via heat transfer through the base of the humidification chamber 1024 of which at least a portion is thermally conductive.

The respiratory support apparatus 10 comprises a flow generator in the form of a blower 11 which draws atmospheric air or other therapeutic gases through an inlet 27/28 and generates a pressurised gases stream 1034 at an outlet of the blower 11. The outlet of the blower 11 is fluidly connected to an inlet 1036 of the humidification compartment 1022 via connecting conduits 1038 extending to the inlet 1036 of humidification compartment 1022. As the humidification compartment is sealed when closed, the gases stream 1034 entering the inlet 1036 pressurises the compartment and gases flow into the open gases inlet 1037 of the humidification chamber 1024. It will be appreciated that in alternative embodiments, the inlets 1036, 1037 of the compartment 1022 and chamber 1024 may be sealingly connected by a connector or other sealing configuration.

The pressurized gases stream passes through the humidification chamber 1024 and exits via gases outlet 1040 of the humidification chamber 1024. In this example embodiment, the gases outlet 1040 of the chamber 1024 is sealingly connected to or sealingly engaged with an outlet 1041 of the humidification compartment 1022 as shown. It will be appreciated that in alternative embodiments, the outlets 1040, 1041 of the compartment 1022 and chamber 1024 need not be sealingly connected by a connector or otherwise sealingly engaged. In the embodiment shown, the outlet 1041 of the humidification compartment 1022 is fiuidly connected via connectors and/or conduits to a patient interface for delivery to a patient P. The patient interface 18 typically comprises a flexible gases conduit 23 coupled at one end to the main gases outlet of the respiratory apparatus 10 and to a user interface 1046 at the other end.

In an example embodiment, the humidification chamber 1024 is received and retained within a complimentary enclosed and sealable humidification compartment 1022 formed in the housing of the respiratory apparatus 10. However, it will be appreciated that the humidification chamber 1024 could alternatively be received and retained in an open or exposed compartment or on a support platform comprising the heater pad 1028 in alternative embodiments with the gases inlet of the chamber being connected to the blower outlet by conduits and/or connectors and the gases outlet of the chamber being connected by conduits and/or connectors directly or indirectly to the patient interface 18.

Some examples of respiratory support apparatuses are disclosed in International Application No. PCT/NZ2016/050193, titled “Flow Path Sensing for Flow Therapy Apparatus”, filed on Dec. 2, 2016, and International Application No. PCT/IB2016/053761, titled “Breathing Assistance Apparatus”, filed on Jun. 24, 2016, which are hereby incorporated by reference in their entireties. Examples of configurations of respiratory support apparatuses that can be used with aspects of the present disclosure are discussed in further detail below.

### Valve Module

FIG. 5 shows a first configuration of a valve module 4001 that could comprise part of a respiratory support apparatus in accordance with this disclosure. The valve module 4001 controls the flow of oxygen and/or other gases entering the gas flow path of the apparatus 10, and enables the apparatus 10 to regulate the proportion of oxygen entrained in the airflow. The valve module is formed as a modular unit for ease of manufacture, assembly, servicing, or replacement, for example in the event of malfunction, routine maintenance, or future upgrade/improvement.

The valve module comprises a flow control valve 4003 that is arranged to control a flow of gas through a valve manifold 4011. The valve is arranged to control a flow of gas into part of the apparatus. For example, the valve may be arranged to control a flow of gas to a filter module 1001. Alternatively, the valve 4003 may be arranged to control a flow of gas to another part of the apparatus. The valve module 4001 and filter module 1001 are positioned upstream of the blower 402 and motor and/or sensor module 400.

The flow control valve could be a solenoid valve, could be motor-driven, or could be piezo-operated for example.

In a solenoid valve, the valve member is actuated between open and closed positions. The solenoid valve may be a proportional valve. The extent of gas flow through the valve (i.e. due to the size of the valve opening) is relative to the electrical current supplied to the valve.

Alternatively, the solenoid valve may be controlled with a modulated input signal, so that the valve is modulated between open and closed positions.

The valve 4003 could be a needle valve, plunger valve, gate valve, ball valve, butterfly valve, globe valve, etc. The valve may be of the pressure compensated type.

In some configurations, the valve is a normally-closed valve; that is, the valve is closed when powered off. That will prevent a connected gas supply line continuously releasing oxygen or other gas when the apparatus is powered off. In some alternative configurations, the valve is a normally-open valve.

A filter may be provided inside the valve manifold gases inlet 4017 inlet to prevent the introduction of dust or particulates into the valve.

The valve module 4001 is located at the start of the flow path of the apparatus. If the valve 4003 was to be obstructed (i.e. by dust, particulate, etc.) such that it would be held open, excess pressurized oxygen or other gas would ‘dump’ through the ambient air entry opening(s) in the valve carrier. This would prevent any excess pressure reaching the patient. As such, the system may be considered inherently pressure limited without the use of a pressure relief valve.

The apparatus may simultaneously draw in gas from the gases inlet of the valve manifold and ambient air, or the pressurization of gas from the gases inlet may force that gas through the filter. The gases will exit the valve module and enter the gases inlets in the filter. The apparatus may be configured such that the gas from the gases inlet and the ambient air are dynamically entrained/mixed in the apparatus prior to being delivered to the gases outlet of the apparatus. The valve module may draw in gases from an expiratory conduit, as will be described further below, for example via the alternative supply. The expiratory conduit may be coupled to the alternative supply to draw in the recirculated gases i.e. gases from the expiratory conduit. The filter module may comprises a HEPA filter or other filters that can be used to filter out microbes such as for example viruses, and/or other substances such as medicament and/or particulate matter. The filter module may comprise multiple layers of filters to filter out various substances from the recirculated expired gases. For example, one or more layers could be configured to filter out particulate matter, pathogens and/or microbes. For example, the filter module may comprise stages of filtering e.g. a first stage for filtering particulate matter, a subsequent stage for filtering liquid condensate, a subsequent stage of filtering microbes, and a subsequent stage for filtering gases. The filter could comprise a single barrier e.g. bidirectional, hydrophobic media to prevent passage of airborne and liquid borne microbes, pathogens and particulate matter.

The filter module and valve module described herein may provide varying gas flow paths for the apparatus. For example, the valve module may control the flow of oxygen entering the gas flow path of the apparatus, via the valve module and filter module. Alternatively, the valve module may be bypassed by means of direct connection of an alternative oxygen source to the filter module by the first sub-compartment gases inlet (inlet 27 of FIG. 13 for example). This may be practical in circumstances where a user may wish to manually adjust the oxygen supply (i.e. such as by a wall supply rotameter).

In the configurations shown, the apparatus 10 receives oxygen by at least one of the following: via the valve module (for automatic oxygen regulation by the apparatus), or via the alternative gases inlet provided on the top of the filter (allowing attachment of a manually adjustable oxygen supply—i.e. such as by a wall supply rotameter).

The various configurations described are exemplary configurations only. Any one or more features from any of the configurations may be used in combination with any one or more features from any of the other configurations.

Both a CPAP/BiLevel apparatus as per FIGS. 1C/1D for example, and a High Flow apparatus as per FIGS. 1A/1B for example, are single limb systems in that there is a single gases flow path between the flow generator and the patient interface, namely the inspiratory gases delivery conduit. The following disclosure relates to a respiratory support apparatus (which could be any respiratory support apparatus such as a CPAP/Bilevel apparatus or a high flow apparatus for example) having dual limbs, one of the limbs comprising an expiratory limb along which exhaled gases are forced, and optionally recirculated. The flow generator is used for both providing positive pressure (i.e. positive flow of inspiratory gases toward the patient) and in some embodiments for generating suction forces via the creation of negative pressure in the expiration tube (to cause a negative flow of expiratory gases away from the patient). The expiratory gases may be cleaned, for example by the filter module, and recirculated to the patient.

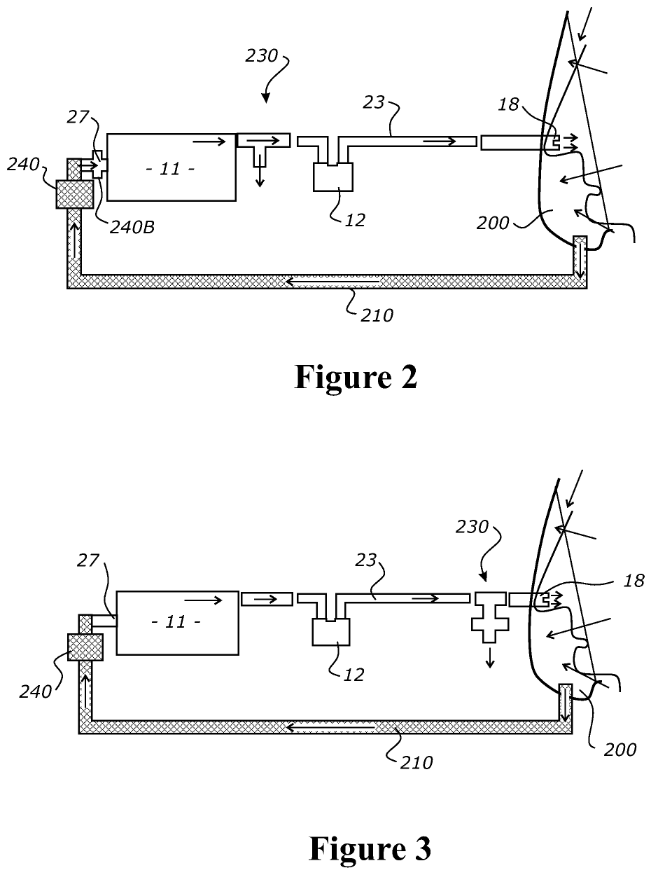

With reference to FIG. 2, in this embodiment a respiratory support apparatus is shown schematically, and is provided in line with the apparatus 10 of FIG. 1A, with like features being given like references. The apparatus of any of FIGS. 2 to 24 can have any or all of the features of the apparatus 10 of FIG. 1A. Broadly, the respiratory support apparatus 10 can be considered to comprise a flow generator in the form of a blower 11, a patient or inspiratory conduit 16, and a first patient interface comprising a non-sealing nasal cannula comprising prongs 18, wherein the patient conduit 16 comprises an inspiratory gases flow path between the blower outlet 21 and the patient, configured to deliver a high flow of gases to the patient interface. In this example, the apparatus 10 also comprises humidifier 12. The humidifier 12 may be integrated with the flow generator 11, that is the humidifier 12 and flow generator 11 are housed in a common housing. Alternatively the humidifier may be a separate component that may be fluidly coupled to the flow generator 11, that is, the humidifier 12 and flow generator 11 may be in separate housings. The flow generator 11 in this example comprises a single blower, an inlet comprising ambient air inlet port 27, and outlet port 21. The nasal cannula engages in an unsealed manner with the nostrils of the patient.

Apparatus 10 further comprises a face shield or cover, being a second patient interface 200 which in this example comprises a full face mask which extends over at least the patient's nose and mouth. The face shield or cover includes a headgear that is used to secure the face shield or cover to the patient. The face shield preferably does not form a seal with the face or airways of the patient, thereby preventing pressure being generated within the patient's airways. The unsealed nature of the face shield or cover allows high flow respiratory support to be delivered to the patient. The face shield 200 in this example is located around, but does not seal against the nose and mouth such that gases from the ambient environment may be drawn into the volume between the face shield 200 and the patient's face, as indicated by the arrows in FIG. 2. Nonetheless, gases exhaled by the patient are not exhausted to the ambient environment. Examples of a face shield in accordance with this disclosure could be a full face mask which extends around the nose and mouth, a total face mask which also extends around the eyes, or a head mask or helmet which also extends around the patient's head, for example around the crown, or rear of the head. The face shield may comprise a hood configured to extend over and around the entire head of the patient, or may comprise a frontal screen that extends over the front of the patient's head.

In accordance with this disclosure, an expiratory flow path (i.e. an expiratory circuit) is provided extending between the face shield 200 and the inlet 27 of the flow generator 11. Expired gases exhaled by the patient are forced along the expiratory flow path by the flow generator 11, as will be described below. The expiratory flow path comprises one or more expiratory ports in the face shield 200 and one or more expiratory conduits 210 and suitable conduit connectors, to connect the expiratory conduit(s) 210 to the expiratory port(s) patient interface 200 and flow generator inlet 27. The expiratory conduit 210 may comprise a breathable portion or limb that allows passage of water vapour without allowing passage of liquid water. The face shield 200 may be shaped so as to help direct expiratory gases within the volume defined by the face shield 200 toward to the or each expiratory port. For example, the face shield 200 may comprise one or more angled walls, inclined towards the or each expiratory port. The face shield 200 functions as a face cover that covers at least part of the patient's face to prevent exhaled gases from passing to the ambient environment. For example, exhaled gases from the mouth and nose may be redirected from the face shield back towards the patient's face.

In a typical high flow apparatus, for example as shown in FIGS. 1A/1B, the patient can exhale via their mouth, the exhaled gases being exhaled directly to the external environment. The flow generator 11 in this example is configured to generate a high flow of gases by drawing in ambient air through inlet 27, pressurizing these for example via a blower comprising one or more rotating impeller(s), where the high flow of gases generated exit the flow generator 11 via the outlet 21 and are delivered to the patient via the patient conduit 23. The or each impeller may be a low inertia impeller, allowing it to accelerate to the appropriate speed quickly. The flow generator 11 generates a positive flow of gases in a direction towards the patient. There is therefore positive pressure at the flow generator outlet 21 and negative pressure at the flow generator inlet 27 This negative pressure or suction may draw ambient air into the flow generator 11. The current disclosure uses this negative pressure or suction to create a negative pressure or suction in the expiratory conduit 210, to draw expiratory gases along the expiratory conduit 210 towards the flow generator 11, from inside the volume defined by the face shield 200. Those expiratory gases can be cleaned and emitted to the ambient environment, or cleaned and recirculated to the patient through the flow generator 11. The expiratory gases are cleaned to remove, or at least reduce, unwanted components of the expiratory gases flow. The cleaning function is performed for example by the filter module and/or CO₂ scrubber. Although the face shield 200 does not seal against the patient's face, the negative or reduced pressure in the expiratory conduit 210 is sufficient that expiratory inside the face shield 200 are drawn through the or each exhalation port and the expiratory conduit 210 without being emitted to the ambient environment. A respiratory support apparatus in accordance with this disclosure thus controls what happens to the exhaled gases, and in particular prevents, or at least reduces, any aerosols or other heavier components in the exhaled gases from reaching the ambient environment. Further detail of an apparatus in accordance with this disclosure is provided below.

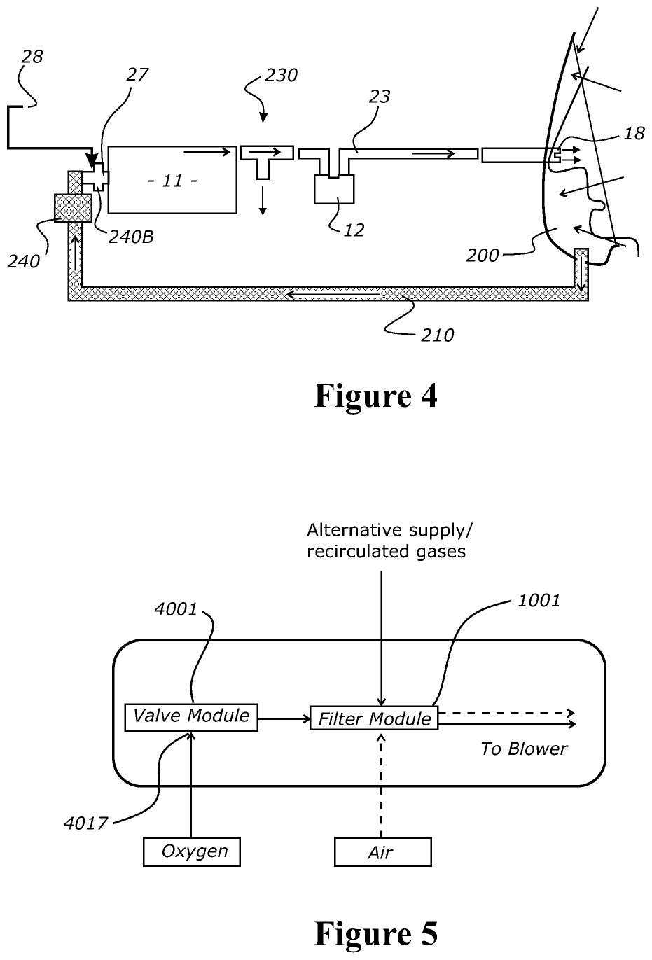

The apparatus 10 is provided with a leak port or valve 230 in the inspiratory gases flow path between the flow generator 11 and the patient interface 18. In the example of FIG. 4, the leak port or valve 230 is located adjacent the flow generator outlet 21. The leak port or valve 230 is configured to allow an amount of the gases flow generated by the flow generator 11 to leak from the inspiratory flow path such that the flow rate of inspiratory gases delivered to the patient interface 18 is less than the flow rate of inspiratory gases generated by the flow generator 11 at the outlet 21. This ensures that the suction forces generated by the flow generator 11 are sufficient to draw all of the expired gases along the expiratory conduit 210. The suction rate within the expiratory conduit 210 is equivalent to the delivered flow rate generated by the blower. The negative flow rate is equal to the positive flow rate generated by the blower. The suction rate (i.e. negative flow rate) in the expiratory tube is greater than the total flow rate delivered to the patient, since some of the positive flow (i.e. flow provided toward the patient) is vented out of the leak port, such that the flow rate reaching the patient is reduced. The total suction flow rate (i.e. negative flow rate) is greater than the total flow rate delivered to the patient through the nasal cannula.

The leak port or valve 230 may also allow the system to compensate for extra exhalation by the patient. Preferably the leak port reduces the flow rate delivered to the patient. The leak port reduces the total flow and total volume of gases delivered to the patient to the patient. The leak port may comprise a tubular body that includes one or more openings. The leak port may include only a single opening.

With reference to FIG. 6, an example of a suitable leak port can be seen in PCT/NZ2016/050101 filed 27 Jun. 2016, the entire contents of which are incorporated herein by reference. In the illustrated configuration, the leak port 1100 is disposed in-line with inspiratory conduit 23, such that the exhalation port 1100 has a first end 1102 that is in fluid communication with one portion of conduit 23 and a second end 1106 that is in fluid communication with another portion of conduit 23.

The leak port 1100 includes an elongate body 1101 having a hollow portion extending through a longitudinal axis 1103 of the port 1100, defining a lumen 1105 through which gases may flow. The port 1100 includes a first end portion 1102, a center portion 1104, and a second end portion 1106.

The first and second end portions 1102, 1106 may include a 22 mm male taper 108 and 15 mm female taper 110 nested within the 22 mm male taper 108 to enable connection to various conduits 23. The 15 mm female taper 110 can be used to connect to a tracheostomy tube. Other connection formats can be included in embodiments of the disclosed port 1100 as well. The center portion 1104 includes a ⅛ inch pressure line port 1112 to couple with a pressure sampling line that connects to the flow generator. When the pressure line port 1112 is not in use, it may be closed off with a cap (not shown).

The center portion 1104 of the port 1100 may also include a plurality of vent holes 1114 (also referred to herein as “openings 114”) through which the inspiratory gases flow can be leaked from the inspiratory conduit 23. A shroud 1116 may be positioned over and around the vent holes 1114 to reduce draft from the leaked gases. The shroud 1116 is substantially annular. The shroud 1116 allows venting of the exhaled gases and prevents blockage of the vent holes 1114. Illustratively, the shroud 1116 prevents entrainment of surrounding ambient air within the exhalation stream through the vent holes 1114. The shroud 1116 may also have a 22 mm male taper at an outside surface 1120 providing structure to which an external filter (not shown) can be attached.

Leak rate from the leak port of valve may be between 10 L/min to 50 L/min depending on the driving pressure from the flow generator. The leak port or valve is configured to leak out at least 20 L/min in normal use.

In some examples, the leak port or valve 230 may be configurable or adjustable (for example manually, or via a controller of the apparatus 10) so as to allow the amount of leak, or the proportion of the gases flow that is leaked, to be varied. Such variation can allow for example additional leak to be allowed for a patient who frequently sneezes, or who otherwise has periods of excess exhalation.

In the example of FIG. 2, the flow generator 11 may be controlled to provide pressure therapy. During such pressure therapy the flow generator 11 may be controlled to deliver a gases flow at a pressure of 15 mm H₂O, leading to a flow rate at the flow generator outlet 21 of around 70 L/min. A sealed interface would be used when providing pressure therapy. Alternatively, the flow generator 11 may be controlled to provide high flow therapy, for example, so as to provide a flow rate at the flow generator outlet 21 of around 70 L/min. An unsealed interface would be used when providing high flow therapy.

The leak port or valve is configured to leak gases at around 20 L/min. Around 50 L/min of inspiratory gases are therefore delivered to the patient. However, in the expiratory conduit 210, the flow generator 11 is drawing in expired gases at a flow rate similar to that at the flow generator outlet, namely around 70 L/min. The apparatus 10 is therefore configured such that the flow rate of inspiratory gases delivered to the patient is less than the flow rate of inspiratory gases at the flow generator outlet 21 and inlet 27.

One or more gas cleaners 240 may be provided in the expiratory flow path between the patient interface 18 and the flow generator 11. In this example, the gas cleaner 240 is provided at the flow generator inlet 27. In this example the gas cleaner 240 comprises a CO₂ scrubber 240A and a gases filter 240B. The gas cleaner 240 is configured to clean the expiratory gases at least to an extent where aerosols in the expiratory gases are reduced before the expiratory gases are emitted to the ambient atmosphere and/or recirculated to the patient.

The filter may comprise a single filter material, or a combination of filter materials. The filter may be a multi-stage filter, each stage being configured to filter different contaminants from the gases flow. The filter may comprise a HEPA filter and/or an EPA filter. For example, the filter may be a E12 class or higher. The filter includes a gases path through it and connectors on it to allow the filter to be connected into the expiratory gases flow path.

The CO₂ scrubber may also include a system to draw out CO₂ and leave other gases in the gases flow (besides gases and particles that are removed from scrubber). The scrubber may also include other components or other additives to remove toxic gases or other harmful substances from the gases flow. The CO₂ scrubber unit may also include a disinfection unit or a separate disinfection unit adjacent (upstream or downstream) to the CO₂ scrubber to disinfect recirculated gases.

Whether or not the cleaned expiratory gases are emitted to atmosphere and/or recirculated may be controlled by a controller of the apparatus 10, and a valve arrangement may be provided to vary or control the proportion of the cleaned expiratory gases that are emitted to atmosphere and/or recirculated. It is envisaged that 100% of the cleaned expiratory gases are emitted to atmosphere, or recirculated.

Optionally the expiratory gases path may include a heater, such as a heater wire within it, to heat the exhaled gases and prevent or reduce condensation in the expiration path. The flow generator unit can supply power to the heater. Optionally the filter may be heated, for example, with a heater wire going through the filter or a heater around the filter to prevent or reduce condensation within the filter.

The apparatus is preferably a recirculated system where the exhaled gases from the patient are scrubbed of CO₂ and then pumped back through the system and recirculated to the patient in the inspiratory gases flow through inspiratory conduit 23. The gases are filtered to filter out harmful components, such as microbes e.g. viruses etc. Preferably the gases are maintained in a closed system in order to prevent exhaled gases e.g. aerosols, from escaping from the system, thus minimizing the likelihood of spreading the infection. An aspect of this disclosure is therefore an apparatus that is a closed system where exhaled gases cannot escape, and where exhaled gases are cleaned and recirculated to the patient.

In an alternative system the exhaled gases may be scrubbed, then filtered and then vented to atmosphere.

Referring now to FIG. 3, another embodiment of a respiratory support apparatus 10 in accordance with this disclosure is provided. This embodiment is similar to the FIG. 2 embodiment, except that the leak port or valve 230 is in a different part of the inspiratory flow path, namely downstream of humidifier 13 and adjacent the patient interface 18. In this embodiment, the leak port or valve 230 may be provided on the patient interface 18 or on the face shield 200, as long as the inspiratory flow is leaked prior to the flow being in the sealed cavity defined inside the face shield 200.

Referring now to FIG. 4, another embodiment of a respiratory support apparatus 10 in accordance with this disclosure is provided. This embodiment is similar to the FIG. 3 embodiment, except that the flow generator 11 also comprises a supplementary gases inlet 28, for example for providing oxygen into the inspiratory gases flow. This embodiment may comprise a gases inlet valve module 4001, for example as described above with reference to FIG. 6. Such a valve module can be configured to control the amount of oxygen and ambient air being received in the flow generator 11. In this example, the expiratory conduit 210, and gases cleaner 240, can be fluidly connected to the valve module 4001.

In these High Flow examples, the expiratory gases are located at or adjacent the face shield 230, in the cavity or volume defined between the face shield 230 and the patient. The face shield 230 does not seal with the patient's face, but covers at least the patient's nose and mouth, and directs expiratory gases from the patient to the leak port 230.

### Pressure Support Respiratory Apparatus

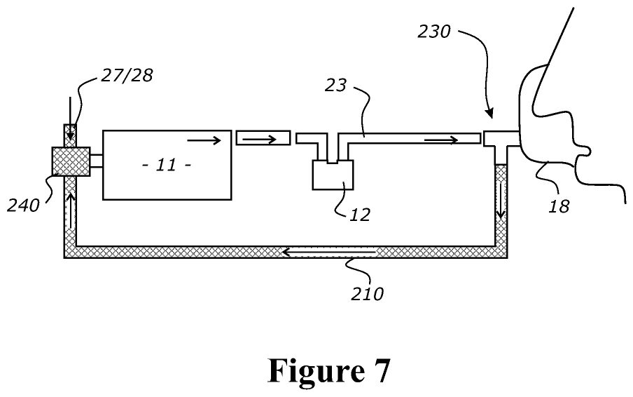

In accordance with this disclosure, and with reference to FIG. 7, another embodiment of a respiratory support apparatus comprises a pressure support apparatus such as a CPAP or BiLevel system for example, with features similar to those of FIGS. 1C/1D, which is controlled according to attempting to deliver a gases flow at a desired pressure to the patient. Such an apparatus comprises a patient interface 18 that is sealed against the face of the patient, in this example sealed around the nose and mouth. In such an apparatus, face shield 200 is not required, because the patient interface 118 is sealed against the patient's face, which therefore prevents or at least resists the leaking of expiratory gases to the ambient environment.

In the pressure support example, the exhalation port is provided in the patient interface 18, with the expiratory conduit 210 being pneumatically connected to the patient interface 18 via the exhalation port. The exhalation port is large enough such that when the patient exhales, the exhaled gases travel through the exhalation port and out of the expiratory conduit 210. Optionally the pressure support system of FIG. 7 may include an additional supplementary gases inlet that allows supplementary gases to be provided to the blower. The supplementary gases may be oxygen. The supplementary gases may be similar to that shown in FIG. 4. The expiratory conduit 210 may be directly connected to an exhalation port of the patient interface. Alternatively, the expiratory conduit 210 may be positioned so that the inlet end of the expiratory conduit 210 is adjacent a leak port or leak region of the patient interface, to capture at least some of the expiratory gases.

A problem that can be solved with the High Flow or the Pressure Support Apparatus is to conserve oxygen and recirculate oxygen to minimize wastage of oxygen delivered to the patient. Recirculated flow from the patient interface via the exhalation port is advantageous because oxygen delivered to the patient is recirculated via the expiratory port and expiratory conduit 210. For example, in a traditional CPAP system some of the oxygen is wasted as it travels out of the leak port in the patient interface as a bias flow. Bias flow is the flow of gases needed to maintain gas flow at the desired pressure or flow rate, taking into account leakage. In this recirculated system that oxygen or excess oxygen not breathed in by the patient is captured and recirculated. In the system shown in FIG. 7 the mask is a sealed mask that seals around the patient's airways. The mask 18 may be a nasal mask or a full face mask. The exhaled gases enter the mask 18 and are routed out through the expiratory conduit. The exhaled oxygen is recirculated and delivered to the patient. This allows conservation of oxygen. The expiratory conduit also has a scrubber e.g. a CO₂ scrubber to remove CO₂. The expiratory conduit may also include a disinfection unit adjacent the CO₂ scrubber to disinfect recirculated gases before being delivered to the patient. The outlet of the expiratory conduit is coupled to a filter e.g. a HEPA filter that is upstream of the inlet of the flow generator 11 (i.e. blower 11). The filter is configured to filter out particulates, microbes and other material. The filter helps to provide clean, recirculated gases to the patient at a desired pressure.

In the pressure support apparatus example, the blower of for example a CPAP apparatus, creates a positive pressure i.e. delivers a positive flow to the patient via the inspiratory conduit, and creates a negative pressure in the expiratory conduit to suction out gases through the leak port in the mask.

The leak port in the pressure support apparatus may be in the inspiratory path adjacent the mask or in the mask. The leak port may be a single opening or may have multiple openings.

In each of the above examples, the expiratory port in either the face shield 200 or the patient interface 18 may comprise one or a plurality of port openings, and/or a valve.

In each of the above examples, the flow generator outlet 21 comprises an outlet connection which can comprise one or more connectors enabling the inspiratory conduit to be connected to the outlet 21. The outlet connection can comprise, for example, a male or female push fit connector on which a corresponding male or female push fit connector on the end of the inspiratory conduit is configured to be received.

In each of the above examples, the flow generator inlet(s) 27/28 comprises an inlet connection which can comprise one or more connectors enabling the expiratory conduit to be connected to the flow generator 11. The inlet connection can, for example, comprise a male or female push fit connector on which a corresponding male or female push fit connector on the end of the expiratory conduit is configured to be received.

The inlet connection can comprise an inlet manifold that provides a pneumatic connection between the inlet(s), the expiratory conduit, and the flow generator 11. The manifold could comprise the valve/filter module 4001/1001.

In FIGS. 2 to 4 arrows are shown in the face shield. These broadly and generally indicate some example gases flows within or around the face shield, for example exhalation flows from the nose, mouth and eyes. Some of the arrows indicate, in FIGS. 2 to 4 some gases flow into the face shield 230 around the seal provided between the face shield and the patient's head. In the case of pressure support embodiment of FIG. 7, the arrows illustrate that the seal provided by the patient interface 18 is unlikely to be a perfect seal.

The figures also indicate some flow rates in different parts of the apparatus. These are examples only.

When or if respiratory apparatus provides high flow therapy, for example NHF the apparatus of FIGS. 4 and 7 can provide a useful benefit of oxygen conservation. NHF therapy can typically use a relatively high amount of oxygen, due to the high gases flows used in the therapy. Recirculating flow and capturing the expired gases as well as the leaked gases from the leak port helps to capture oxygen, that is recirculated.

It is also envisaged that a respiratory support apparatus as described above, could be provided without the leak port or valve. In such an example, a respiratory support apparatus configured to provide a gases flow to a patient could comprise:
a. a flow generator configured to generate the gases flow and comprising an inlet and an outlet, the flow generator being configured to draw inspiratory gases in through the inlet, and to deliver the gases flow through the outlet;
b. a patient interface configured to be mounted on the patient and to receive the gases flow from the flow generator outlet to deliver the gases flow to the patient;
c. an expiratory conduit configured to extend between, and be in fluid communication with, the patient interface and the flow generator inlet, to provide a flow path for expiratory gases between the patient interface and the flow generator inlet;
d. wherein the generation by the flow generator of the gases flow also generates forces in the expiratory conduit, such that the flow generator forces expiratory gases along the expiratory conduit from the patient interface.

We also provide, in an aspect of this disclosure, a respiratory support apparatus configured to provide a gases flow to a patient comprising:
a. a flow generator configured to generate the gases flow and comprising an inlet and an outlet, the flow generator being configured to draw inspiratory gases in through the inlet, and to deliver the gases flow through the outlet;
b. a breathing circuit in fluid communication with the outlet of the flow generator and the inlet of the flow generator, wherein the breathing circuit defining a closed gases flow path to deliver gases generated by the flow generator to the patient, and to recirculate the exhaled gases from the patient to the inlet of the flow generator.

In this disclosure the patient interface comprises an inspiratory gases flow delivery interface configured to deliver the inspiratory gases flow to the patient. The patient interface may be sealing or non-sealing, primarily in dependence upon the type of flow being delivered to the patient, and configured to prevent or at least reduce, gases, whether inspiratory or expiratory gases, from leaking from the patient to atmosphere. The face shield or second patient interface is only required in a high flow apparatus which uses a non-sealing patient interface, and comprises a non-sealing interface configured to direct expiratory flow in a desired direction. The patient interface and the face shield could be integral parts of the same component, separate components, or could be separate components that are configured to be mounted or connected together to form an assembly. The patient interface is coupled to the inspiratory conduit and may engage the patient's airways in a sealed or an unsealed manner.

The face shield or second patient interface may be applied to the patient and covers the first patient interface and part of the patient's head. In the case of a sealing patient interface, this is connected directly to the expiratory conduit such that exhaled gases are drawn through the expiratory conduit.

The face shield or second patient interface, if provided, is coupled to the expiratory conduit such that exhaled gases are drawn through the expiratory conduit.

In this disclosure the flow generator can generate sufficient forces in the expiratory conduit to force expiratory gases along the expiratory conduit, to the flow generator inlet. There must be sufficiently low pressure in the expiratory conduit, relative to the pressure at least in the patient interface, for the expiratory gases to flow along the expiratory conduit. These forces could be suction forces generated by an at least partial vacuum or a reduced pressure, relative to the pressure in other parts of the apparatus and/or ambient pressure. There should be a pressure, somewhere in the gases flow path, sufficient to create gases flow from the patient towards the flow generator inlet. The pressure in the expiratory conduit may be less than the pressure in any inspiratory conduit, but not necessarily below atmospheric pressure.

Where the respiratory support apparatus comprises a high flow apparatus, the leak port leaks out some of the flow generated by the flow generator such that the flow delivered to the patient is less than the flow that is suctioned out through the expiratory conduit. This ensures that expiratory flow due to suction/negative pressure is more than the delivered flow. This can help to capture all or most of the exhaled gases, and may also draw in some ambient air.

Where the respiratory support apparatus is a pressure support apparatus, the leak port may be the only outlet for the exhaled gases. The leak port serves to vent (i.e. allow escape) of exhaled gases since there is a pressure gradient from high pressure in the inspiratory conduit toward the leak port and out into the expiratory conduit. The expiratory conduit is connected to the leak port such that any suction forces in the expiratory conduit ensures pressure in the expiratory conduit draws exhaled gases through the leak port into the expiratory conduit. The expiratory conduit pressure is less than the inspiratory conduit pressure (i.e. the pressure in expiratory conduit may be the same magnitude but an opposite direction) therefore a pressure gradient is created and exhaled gases pass from the leak port into expiratory conduit.

A respiratory support apparatus can be arranged to deliver a breathable gas from a gas source to the patient, in addition to, or as an alternative to, delivering ambient air. For example, some respiratory support apparatus can receive oxygen from an oxygen source. The oxygen source could be a pressurised oxygen cannister or the like, or could be a wall source of oxygen as might be provided in a hospital for example. It can be desirable to try to conserve the source of gas by minimizing or at least reducing wastage of that gas, not least because oxygen can be relatively expensive.

Some respiratory support apparatus comprise one or more leak ports in the breathing circuit between the flow generator and the patient, in an inspiratory limb and/or expiratory limb of the breathing circuit. Such leak ports can form an intentional leak path from which breathing gases or expiratory gases can leak. For example in a CPAP respiratory support apparatus, the leak port provides a leak for expired gases to pass out of. However, some of the breathing gases can also leak out of the leak port. In prior art leak ports, the amount of flow leaked through the leak port increases as the pressure output from the flow generator increases, increased pressure generating more gases flow, hence more leak flow through the leak port. This can cause, for example, undesirable wastage of oxygen (or any other gas from a gas source) especially as the pressure output/flow output from the flow generator is increased. There is a need to increase oxygen conservation and reduce waste of oxygen.

In aspects of this disclosure a respiratory support apparatus is provided that comprises a leak port configured to provide a controlled leak of the gases flow between the flow generator outlet and the patient interface; the leak port being configured:
a) to provide variable occlusion of the leak flow path through the leak port, the occlusion increasing as the pressure and/or flow rate of the gases flow through the breathing circuit increases; and/or
b) such that the leak flow through the leak flow path is substantially constant across a range of pressures and/or flow rates through the breathing circuit; and/or
c) such that increased gases flow to the patient, or from the flow generator, does not lead to increased leak flow (i.e. increased leak).

The occlusion of the leak path may be varied passively, without a controller, or actively, with a controller. For example, a controller may use an electronic signal. The controller may receive, or generate, a control signal indicative of the pressure and/or flow rate of the gases flow to the patient.

Such a leak port can be used with an apparatus as described above with reference to FIGS. 1 to 7, where expiratory gases are captured and recirculated to prevent aerosol dispersion, and/or to conserve oxygen (by recirculation). This aspect of this disclosure is directed to a leak valve/leak port where the leak is controlled by the flow generator, preferably such that the leak is substantially constant over the entire operating range of pressures and/or flow rates of the flow generator. The controlled leak may be useful when providing high flow therapy, due to the high flow that can otherwise lead to relatively high oxygen loss and wastage.

The present disclosure proposes a solution or at least an improvement by controlling the amount of leak through the leak port.

In some examples, the amount of occlusion in the leak port, or the size of the leak path, is adjusted by the flow generator as the pressure or flow output from the flow generator increases. The occlusion level is increased i.e. the size of the leak path is reduced, as the pressure/flow rate of the gases flow increases, such that the amount of leak is controlled across the operating pressure. The leak is preferably kept constant across all pressure levels. Therefore, as the pressure/flow output increases the leak path size is reduced or occlusion is increased. This reduces the amount of leak for the particular increased pressure/flow rate output. The reduced leak flow conserves oxygen as less oxygen is lost to atmosphere through the leak path. In other words, with an apparatus in accordance with this disclosure, the leak of oxygen does not increase, or any increase is reduced, even if the pressure/flow rate of the gases flow delivered to the patient increases. This helps conserve oxygen during respiratory support even as pressure/flow rate is varied.

Oxygen can be delivered anywhere in the flow path between the flow generator and the patient, either on the inspiratory path or expiratory path. In some examples, the oxygen is delivered in the inspiratory path, at a position at the flow generator, at the patient interface, or between the flow generator inlet and the patient interface. The oxygen is delivered into the flow generator preferably upstream of the flow generator inlet such that the flow generator can act as a mixer to mix air/recirculated gases and additional oxygen. Alternatively another mixing arrangement can be used e.g. a separate mixer or a static mixer.

In an apparatus as described above with reference to FIGS. 1 to 7, the pressure/flow output increases in order to, for example, provide additional respiratory support to the patient. This would typically be based on the prescription from a clinician. In some instances, the flow/pressure output may be increased as the blower is driven harder to increase the scavenging function of the apparatus, for example for a predetermined period of time. The scavenging function i.e. the suction of exhaled gases from the patient interface along the expiratory conduit and back into the flow generator, is increased by increasing the blower speed (in particular the blower motor speed). This increased blower speed causes an increased output from the flow generator, but also creates more suction in the expiratory conduit. The increased suction in the expiratory conduit improves the scavenging function. The increased scavenging improves exhaled air and aerosol capture, and improves recirculation of oxygen. The controlled leak via the leak port reduces the oxygen lost to atmosphere as the scavenging function is increased. In other words, the variable occlusion leak port compensates, in respect of oxygen loss, for the increased oxygen loss that would ordinarily occur when the pressure/flow rate of the gases flow to the patient is increased.

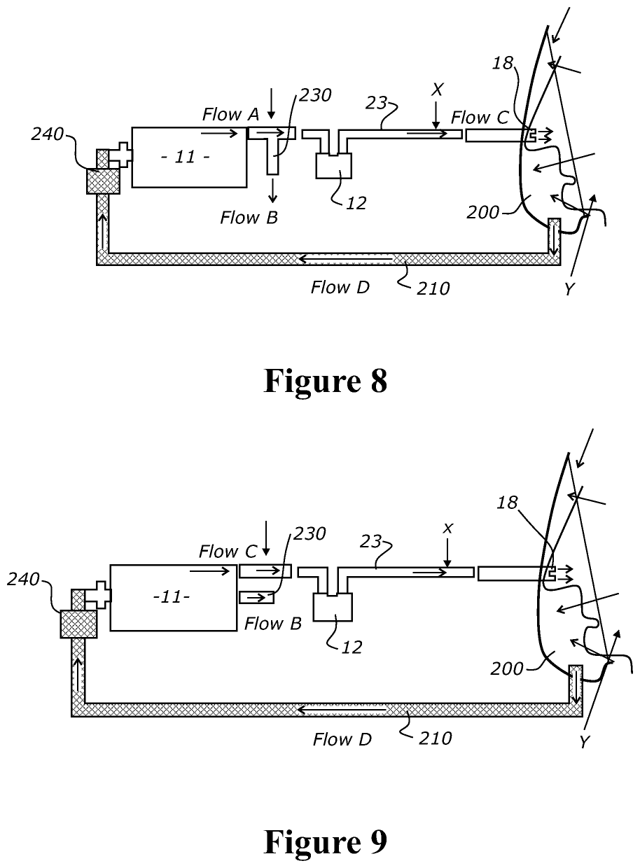

With reference to FIG. 8, an apparatus in accordance with this disclosure is shown which is similar to the apparatus disclosed in FIG. 2 as described above. The leak port is provided at the flow generator outlet, upstream of the humidifier. The breathing circuit of this apparatus scavenges gas exhaled by the patient via the expiratory conduit, as described above. The various flows at different parts of the breathing circuit are labelled in FIG. 8.

FLOW D, in the expiratory conduit, must be sufficient to pull (i.e. draw) in air at the patient interface and to do this it must always be less than FLOW C, being the inspiratory gases flow into the patient interface. An initial estimate for inward flow at the patient's face is:

FLOW D-FLOW C-FLOW B is 20 l/min.

FLOW A (the flow generator gases flow output) is equal to FLOW D, if the input to the flow generator is sealed. In the illustrated example the expiratory circuit is coupled to the flow generator inlet and is sealed to reduce or prevent drawing in ambient air. In some instances, a relatively small amount of ambient air may be drawn in through an ambient air inlet.

Therefore, to create scavenging gas flow around the patient's face, one can divert some of FLOW A, for example via venting the gas flow via a leak port or other vent, and deliver FLOW C to the patient:

FLOW C-FLOW A (which-FLOW D)−FLOW B

FLOW D-FLOW C determines the scavenging inward flow into the flow generator inlet. However, this lowers FIO₂, creating a trade-off of benefits. FIO₂ will rise as FLOW B decreases (because there is less dilution of the gases flow), but lowering FLOW B will also reduce the desirable difference between FLOW D and FLOW C (which difference determined the degree of scavenging of exhaled gas).

The location of the addition of supplemental oxygen into the gases flow also affects FIO₂.

For example, if supplemental oxygen is added at Point X, this will increase FLOW C, and thus reduce the difference between FLOW D and FLOW C, reducing the expiratory gases that are scavenged from the patient.

If supplemental oxygen is added at Point Y, oxygen replaces the ambient air sucked in, by the difference between FLOW D and FLOW C, and does not affect “scavenging” of patient exhalate.

If supplemental oxygen is added to a location outside the face shield (i.e. at one or more locations outside Point Y, near the contact point with the face, but not under the face shield), then the inward scavenging of exhaled gases (ie, FLOW D-FLOW C) is not reduced as above because oxygen simply replaces the entrained ambient air while still contributing (not replacing) direct flow inwards at the shield edge. FIO₂ is still reduced proportionally to FLOW B.

In accordance with this disclosure, to minimize the reduction of FIO₂ for a given addition of supplemental oxygen, FLOW B is controlled, that is, the leak flow from the breathing circuit is controlled, as FLOW A (gases flow from the flow generator) is increased. Resistance through the leak port causes FLOW B to increase with pressure in the breathing circuit. We disclose a number of examples ways to achieve this, as described below.

With reference to FIG. 9, we provide a respiratory support apparatus, similar to that described in FIG. 2, but in which a leak port 230 is provided at the flow generator 11. In this example the leak port 230 may be inside a housing of the flow generator 11, but downstream of the outlet of the flow generator 11. The leak port 230 in this example is a variable leak port configured to variably occlude a leak flow path through the leak port. This arrangement uses the flow generator to control the bypass flow (FLOW B) directly, with the leak port comprising an internal, adjustable leak valve under computer control responsive to measured pressure/flow rate of the gases flow generated by the flow generator. In this example, the leak valve 230 is an active valve, comprising a valve body with a leak passage through which leak flow can pass from the breathing circuit. The valve further comprises valve member movable within the valve body to vary occlusion of the leak passage. A valve actuator, such as a solenoid for example, is provided to move the valve member to variably occlude the leak passage. The valve actuator is controlled by an electronic controller, such as a controller of the flow generator. The leak valve may comprise a solenoid valve.

The controller receives, or generates, a control signal indicative of the pressure and/or flow rate of the gases flow generated by the flow generator 11. The controller uses the control signal to control the actuator to vary the position of the valve member in the valve to vary occlusion of the leak passage. As the pressure and/or flow rate of the gases flow generated by the flow generator 11 increases, the actuator is controlled to move the valve member to increase occlusion of the leak passage. Preferably the occlusion of the leak passage is controlled such that the leak of flow from the leak passage remains constant over the range of pressures and/or flow rates of the flow generator. Consequently, increasing the pressure and/or flow rate of the gases flow from the flow generator, which can be desirable for example to improve scavenging of expiratory gases from the patient, does not also result in an increase in oxygen being lost to ambient. The controller may be the same controller that controls flow (or any other function or parameter of the apparatus) or may be a separate controller. The separate controller may receive flow/pressure measurements and then control the valve to provide a constant leak i.e. control a valve actuator to ensure that the valve member is maintained in a position to maintain a constant leak.

Additional embodiments may be electrically controlled. The leak valve 230 may include a proportional valve that is electrically controlled. The proportional valve may be configured such that as the current/power to the flow generator 11 is increased, the current/power to the proportional valve may be increased. This increased driving current will cause the valve to close as, for example, the blower motor speed is increased. This reduces leak at higher output pressures of the flow generator i.e. leak is substantially constant across the operating range of the flow generator.

This disclosure also discloses providing a variable leak port 230 comprising a passive valve, that is, a valve that automatically varies occlusion of the leak port, without active control by a controller or user. In the examples below, the passive valve varies occlusion using a pneumatic member, exposed to the gases flow, such that the gases flow moves the pneumatic member to vary the occlusion of the leak flow passage through the valve. The pneumatic member is a component, or assembly of components, configured to be exposed to the gases flow and to change its position, shape and/or size to vary by how much the pneumatic member occludes the leak port. The pneumatic member may be substantially rigid, and movably mounted in the leak port so that increased pressure increases the amount of movement of the pneumatic member to vary occlusion. In some examples, the pneumatic member is resiliently deformable, such that the gases flow acting on the pneumatic member changes the position, and/or shape, and/or size of the pneumatic member to vary the occlusion. In some examples, the pneumatic member is exposed to the gases flow via a pressure conduit fed from the gases flow, the pressure conduit being in addition to the valve comprising the leak flow passage.

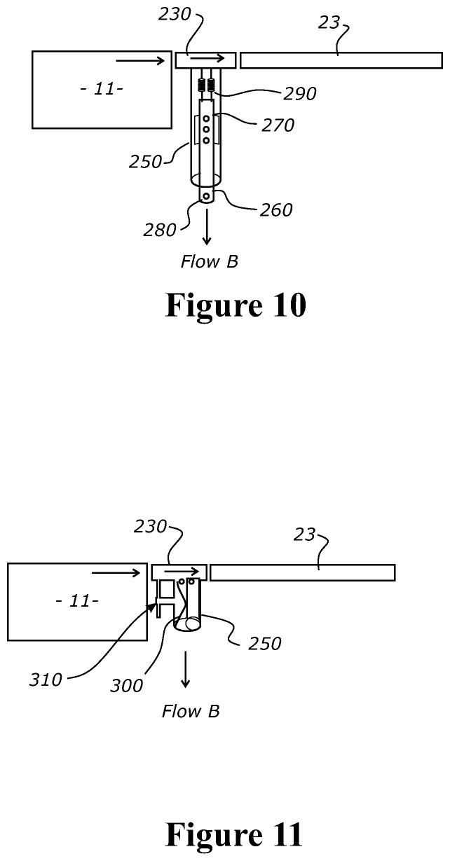

With reference to FIG. 10, one example of a passive valve 230 is provided in which the valve 230 automatically adjusts to provide constant leak across a range of pressures/flow rates of the gases flow. This embodiment provides a valve 230 comprising a valve body 250, which defines the leak flow passage, that includes a biasing element in the form of a spring 290, with a sliding telescoping plunger 260. The spring 290 has a spring constant that is rated to provide a constant leak across all pressures of the gases flow. Spring 290 may comprise any suitable form of spring, including a coil or leaf spring for example, or multiple springs.

Plunger 260 comprises a hollow tube which is concentrically and slidably mounted in the tubular valve body 250. The plunger 260 is provided with a plurality of gas flow apertures 270 along its length which are pneumatically connected to an outlet aperture 280 at a distal end of the plunger 260. Gas can leak from the flow generator or breathing circuit, into the valve body 250 and into the plunger 260. The leak gases then pass along the inside of the plunger 260 and leak from the plunger, and from the valve 230, via the outlet aperture 280.

A biasing element, in this example in the form of a spring 290 (which in this example is a coil spring) is provided to bias the plunger 260 to a position within the valve body 250 such that all of the plurality of apertures 270 are exposed to the gases flow from the flow generator 11. As the gases flow pressure increases, the plunger 260 with multiple apertures 270 slides out of the valve body 250 and as it does so, less apertures 270 are exposed to the gases flow. In other words, as the pressure increases, more apertures 270 are occluded. This means less there are less apertures available for the gases flow to flow out of at higher pressures. The amount of apertures 270 exposed reduces as flow/pressure output from the flow generator 11 increases. This means the leak is controlled directly by the flow generator output. This also ensures that the leak is substantially constant across the operating range of the flow generator 11.

In an alternative embodiment, the plunger 260 could comprise an inlet aperture at one end, rather than outlet aperture 280. In this embodiment the leak gas flows through the inlet aperture, along the plunger, out through the apertures 270, and leaks from the valve via an outlet in the valve body 250.

The relative position between the plunger 260 and the valve body 250 changes as pressure from the gases flow rises, occluding the leak flow passage, and keeping the total FLOW B substantially constant.

With reference to FIG. 11 another embodiment of leak port 230 comprises a passive valve comprising a movable, and in some examples, resiliently deformable, pneumatic member, comprising a component, or assembly of components, that is exposed to the gases flow to vary occlusion of the leak port.

This valve 230 has valve body 250 inside which is a pneumatic member in the form of a diaphragm 300 that is in fluid communication with a pressure tap/pressure line 310 fed from the flow generator outlet or the breathing circuit so as to be exposed to the gases flow. The diaphragm comprises a relatively thin sheet that extends along an exposed part of the leak flow path. In this example the diaphragm 300 is configured to move across the leak flow path through the valve body 250, under influence from the gases pressure from the pressure line 310. As the pressure/flow output increases from the flow generator 11, the pressure in the pressure line 310 increases. This causes the diaphragm 300 to move toward a constricted position that constricts the leak flow passage through the valve body 250. This movement may be via moving the entire diaphragm 300, or by deforming part of the diaphragm 300. The degree of movement varies the degree of occlusion of the leak flow passage. This ensures that leak is constant across flow/pressure output range of the flow generator 11. The leak valve 230 and amount of leak is controlled by the flow generator 11.

In a variant of the valve 230 of FIG. 11, diaphragm 300 may instead comprise a pneumatic member in the form of one or more resiliently deformable bladders inside the leak flow passage of the valve body 250. The bladder inflates as pressure from the gases flow rises, occluding the leak flow passage, and keeping the total FLOW B substantially constant. In this example, the constant leak valve is also controlled by the flow generator output flow/pressure.

In a variant of the valve 230 of FIG. 10, the plurality of apertures 270 in the plunger 260 could comprise a single aperture, whereby movement between the plunger 260 and the valve body 250 varies the amount of the single aperture that is exposed to the gases flow, or at least the amount of the aperture through which leak flow can pass.

It will be appreciated that the leak port 230 could comprise a plurality of ports, each comprising a respective valve.

The pneumatic member of the valve could comprise any component that moves, changes shape, and/or changes size, in response to pressure from the gases flow, to vary occlusion of the leak flow path through the valve body 250. The pneumatic member could be resiliently deformable, or could be substantially rigid, or a combination of the two. For example, the relatively thin, resiliently deformable diaphragm of FIG. 11 could be replaced with a substantially rigid valve member that moves into and out of the leak flow passage under the influence of the pressure from the gases flow.

### No Face Shield

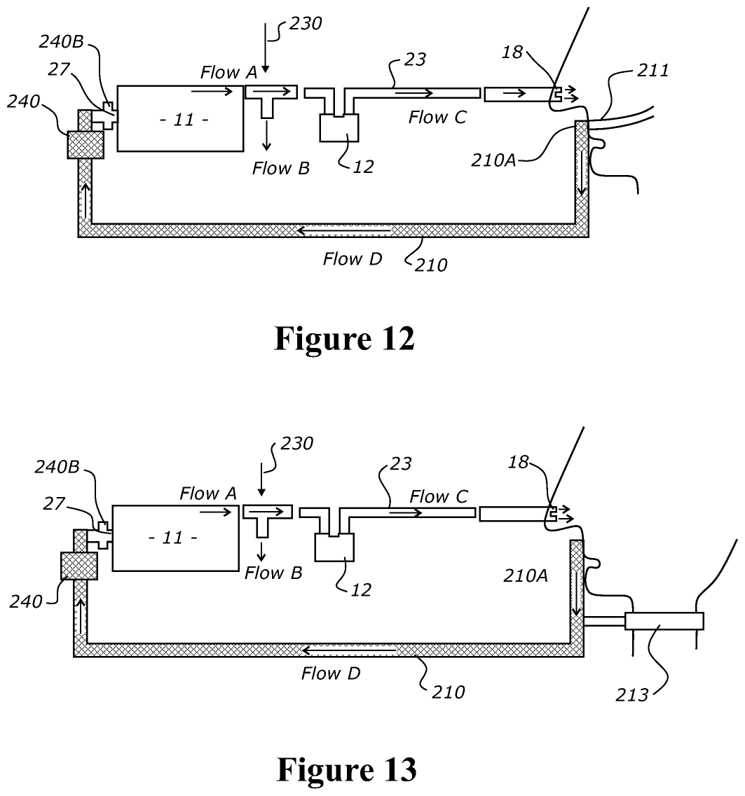

With reference to FIG. 12, we provide a respiratory support apparatus, similar to that described in FIG. 2, but in which face shield 200 is omitted. Face shield 200 in earlier embodiments was configured to at least partially contain exhaled gases so that there is less aerosol dispersion prior to being captured by the expiratory conduit 210.

However, research shows that nasal high flow therapy (without expiratory conduit 210 or face shield 200) can carry a similar risk of aerosol dispersion as normal breathing. Thus, when considering aerosol dispersion, it may not be vital to capture 100% of exhaled gases, as capturing only a portion of exhaled gases can bring aerosol transmission risk down to acceptable levels.

When the recirculating systems described are used without a face shield, some of the exhaled flow can still be captured by the expiratory conduit 210. This means that such apparatus could be used effectively without the face shield 200, and still sufficiently reduce aerosol dispersion. Where a face shield 200 is not used, this can allow patients greater comfort, and flexibility to continue with normal activity such as speaking and eating.

In the embodiment of FIG. 12, the inlet end 210A of the expiratory conduit 210 is located adjacent the mouth, or adjacent the nose and mouth, of the patient to capture exhaled gases and aerosols during exhalation by the patient.

In embodiments without a face shield, it may be preferable to place the inlet end 210A of the expiratory conduit 210 as close to the centre of the nares as possible, provided that the expiratory conduit 210 is distant enough to not interfere with delivered flow prior to the flow exiting the patient's airways.

Expiratory conduit 210 may be provided with a mount to hold at least the inlet end 210A of the expiratory conduit 210 in the desired position adjacent the mouth and/or mouth and nose of the patient.

The mount may comprise a strap 211 attached at or near the inlet end 210A of the expiratory conduit 210. The expiratory conduit 210 may be positioned in the operative position via a mount comprising a lanyard that may be hung around the patient's neck.

Expiratory conduit 210 may be provided with, or comprise, one or more rigid elements configured to increase the rigidity of a section of the expiratory conduit 210, the section being the part of the expiratory conduit 210 closest to the patient. The rigid element(s) provide a scaffolding function to the less rigid expiratory conduit 210.

The rigid element may comprise a spine attached to, or integral with, a section of the expiratory conduit 210 to support that section of the expiratory conduit 210 in a desired location adjacent the patient's mouth or nose. The spine may comprise one or more malleable wires.

Expiratory conduit 210 may comprise a mount configured to be attached to the patient interface 18 or the inspiratory conduit 23 and located adjacent the mouth/mouth and nose. The mount may be a C clip or a U clip, or a snap-ft connector, that retains the expiratory conduit 210.

The expiratory conduit 210 may comprise a mount in the form of a collar 213 that is coupled to the neck of the patient and to which the expiratory conduit 210 is mounted.

Expiratory conduit 210 may comprise a mount configured to be coupled to a headgear or headstrap that, in use, is positioned on the head of the patient and retains the expiratory conduit 210 adjacent the mouth/mouth and nose. The headgear may comprise and one or more of the following:
a) a single headstrap extending from the patient interface 18 around the rear of the head of the patient; or
b) two pairs of side straps, one strap of each pair extending above the patient's ear and the other strap of each pair extending below the ear; or
c) single side straps with a bifurcated rear/crown strap;
d) a chin strap that extends around the chin and back of the head.

The expiratory conduit 210 may be retained by combination of any of the above.

The expiratory conduit 210 may be routed co-axially with the inspiratory conduit 23 for conduit management i.e. so there are not multiple, separate, conduits that may cause a trip hazard, or otherwise become tangled or unwieldy to use. The inspiratory conduit 23 and expiratory conduit 210 may be secured together so that their axes are parallel. Alternatively, the expiratory conduit 210 may be independently routed from the inspiratory conduit 23.

We have described above the use of a face shield 200. The face shield may comprise:
a. Full face mask that covers the nose and mouth, with one or more relatively large leak openings to ensure NHF is provided. The leak opening may be covered with a filter and/or diffuser.
b. Total face mask with a large leak i.e. a leak sufficient that the mask does not create significant pressure when flow is delivered. The total face mask may cover the eyes, nose, and mouth of the patient, or may be a dome that fits around, and encloses the entire head and is secured at the neck of the patient.
c. Hood that covers the head and neck. The hood is preferably unsealed i.e. has one or more leak passages. The hood may be draped over the head and rests on the shoulders of the patient.

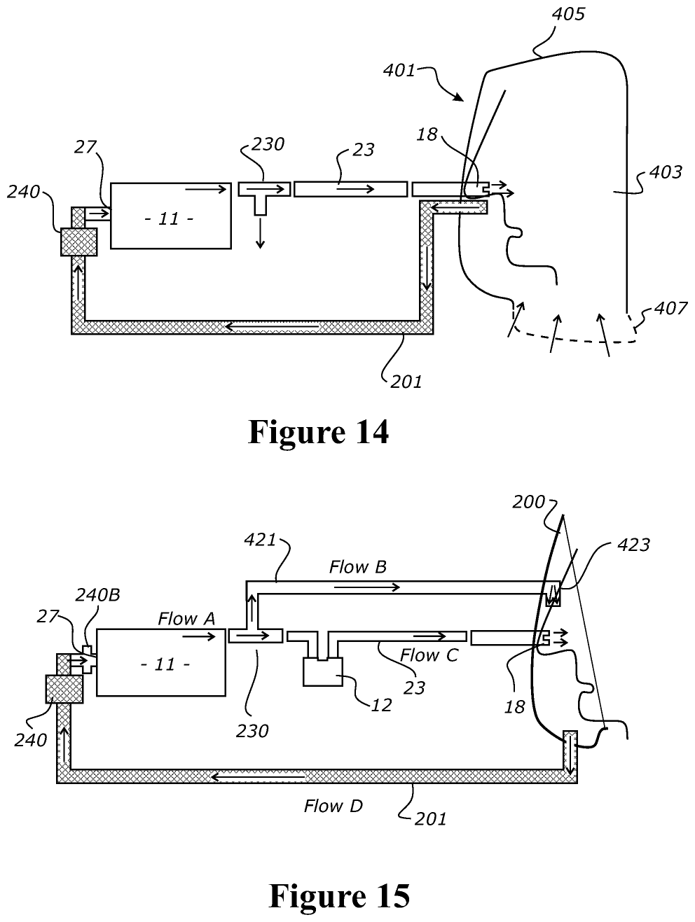

An example hood 401 is shown with reference to FIG. 14 and may comprise a transparent wall 403 that surrounds the patient's head, the hood 401 further comprising a cap 405 that extends over the top of the patient's head, and a lower skirt or collar 407 that extends around the patient's neck. The patient interface 17 may comprise a non-sealed nasal cannula with a manifold 19 and nasal prongs 18 as described above, manifold 19 and prongs 18 being positioned inside the hood 401. The skirt or collar 403 is secured around the patient's neck with a relatively loose fit to allow ambient air to be drawn into the hood 401 in the space between the skirt or collar 403 and the patient's neck.

The expiratory conduit 210 is preferably coupled to an outlet that may be provided in the patient interface 17. The coupling is preferably a low leak pneumatic coupling to ensure that suction is created in the patient interface 17 and suctioned gases are not leaked to atmosphere. If a face shield 200 is provided, multiple suction ports may be provided around the face shield 200 to prevent or at least minimize exhaled gas escaping from certain areas of the face shield 200.

### Reduction of Leak Flow Rates

As described above, the purpose of a high leak flow (an example high leak flow could be 15-20 L/min) is to ensure that the suction flow rate is greater than the delivered flow rate so that as much exhaled gas is captured as possible, in order to eliminate or reduce aerosol dispersion. In an embodiment, it may be desirable to decrease the flow rate of the leak flow. This may mean a smaller portion of exhaled gas is captured, but also that less oxygen-rich air is wasted in the leak flow. This helps to reduce oxygen usage as less oxygen must be added to the system. Consequently the leak port or valve 230 can be configured to provide a desired amount of leak flow that balances these two benefits. If a leak valve 230 is provided, this could be for example a proportional valve that is controlled by a controller of the system to open an amount required to achieve a desired balance of expiratory gas capture vs oxygen retention. This balance can be altered by opening or closing the valve 230.

### Redirecting Leak Flow

We have described above breathing circuits which capture expiratory flow and recirculate it to the flow generator so as to prevent or lessen aerosol dispersion and/or conserve oxygen. As described above, a constant or variable leak flow may be provided upstream of the patient interface to ensure that the expiratory gases drawn away from the patient are greater than those delivered to the patient, making it more likely that most or all of the exhaled gases are captured.

This difference between delivered and captured gas flow means that ambient air is entrained into the flow, while high oxygen content gas may be lost through the leak flow, via port or valve 230. This loss of oxygen can require the addition of supplemental oxygen to the system. We discuss above the relative advantages of adding the supplemental oxygen near the nose and mouth of the patient (for example inside the face shield 200, 401), or outside the face shield 200, 401 but near the contact point with the face of the patient.

A leak flow conduit can be provided and is configured to provide a leak flow path which redirects the leak flow to these same areas: near to the nose and mouth (with or without a face shield) or outside the face shield but near the contact point with the face. The same benefits can be achieved: the difference in delivered and captured flow is now provided by a high oxygen content source rather than ambient gases. The added benefit with this concept is that this source is the leak flow, meaning the oxygen in the leak is recaptured and less supplemental oxygen must be provided.

With reference to FIG. 15, we provide a respiratory support apparatus, similar to that described in FIG. 2, but further comprising a leak flow conduit 421 which is fluidly coupled between the leak flow port or valve 230, and the patient interface 17. The leak flow conduit 421 thus delivers flow leaked from the inspiratory circuit to the patient, that is, inside the face shield 200, 401, or at the least to a position adjacent the patient's nares. The leak flow could be delivered inside the face shield 200, 401, or could be delivered outside of the face shield 200, 401, close to a point where ambient air is entering the face shield 200, 401.

The leak flow conduit 421 can deliver the leak flow at the desired location through a suitable manifold 423, for example at the face shield 200 or hood 401. It would be desirable for the flow to be delivered through the manifold in a direction that doesn't create turbulence that could act to disperse exhaled gases. For example, a manifold positioned above the nares may direct the leak flow downwards around the nose, towards the expiratory conduit 201 below the nose and near the mouth. This may have the effect of guiding gas exhaled from the nose towards the expiratory conduit 210. Alternatively, an annular manifold may be used that encircles the nose and directs the leak flow inwards towards the expiratory conduit 210. Ultimately, the leak flow can be provided in any way that creates a high oxygen surrounding but does not displace exhaled flow. The face shield 200 can be omitted.

As oxygen is used by the patient, and the suction is unlikely to capture the full leak flow, supplemental oxygen may still be required in the circuit, although in a considerably smaller quantity.

In an example, the differential flow between the delivered flow and suctioned flow may be 15 L/min or greater. The leak may be 15 L/min or more, and preferably is between 15 L/min and 20 L/min. The flow generator 11 may be set to outputting a differential flow that is a predetermined L/min (e.g. 30 L/min or more) to achieve high flow therapy.

### Positioning Leak Location

We describe above positioning the leak flow on the patient interface 17 or face shield 200, provided that the leak was outside the face shield 200, for example via leak flow port or valve 230 upstream of the patient.

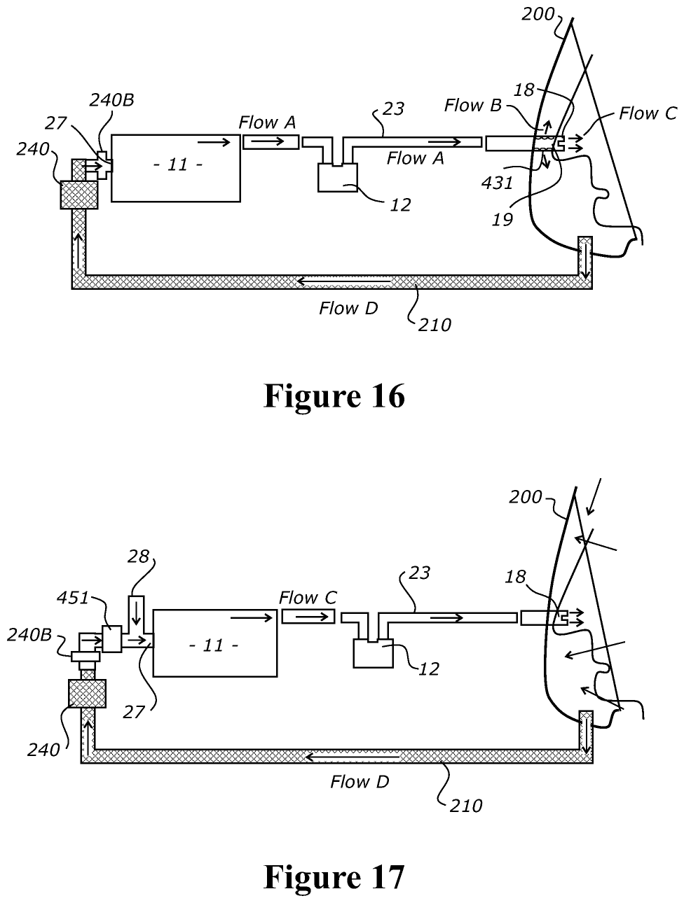

With reference to FIG. 15 we have described reintroducing the leak flow to create a high oxygen environment near the nose and mouth of the patient. With reference to FIG. 16, another embodiment configured to reintroduce the leak flow is provided. In this embodiment the leak flow part or valve 230 is omitted, as is leak flow conduit 421. In this embodiment the patient interface 17 is provided with one or more leak ports 431 configured to leak flow from the inspiratory circuit to a position adjacent the patient's nose or mouth. For example, the leak ports 431 could be provided on manifold 19, as a vent module attached to manifold 19, on a connector connecting manifold 19 to inspiratory conduit 23, or on a cuff at the end of inspiratory conduit 23.

In one embodiment the one or more leak ports are preferably positioned so as not to direct leak flow into the path of delivered or exhaled air, as this could interrupt therapy or increase aerosol dispersion respectively. Instead, the one or more leak ports preferably direct air in one or several other directions (such as above, below and/or to the sides of the nose) to create an oxygen rich environment that increases the overall oxygen content of the flow captured by the suction line.

The positioning of the leak flow near to the nose and mouth avoids the need to retain and route a separate long tube that directs the leaked gases from the leak port or valve 230 located near the flow generator 11 into the face shield 200 or near the expiratory conduit 210. This decreases the number of parts required for this embodiment.

Routing the leak flow into the face shield 200, 401 helps with recirculation of oxygen and helps to conserve oxygen. Oxygen that is leaked from the leak port is captured and recirculated to the inlet of the flow generator 11 and is recirculated through the apparatus. This helps to reduce the need for external oxygen to be supplied from an external oxygen supply, such as an oxygen tank or hospital wall supply.

### O2 Concentrator

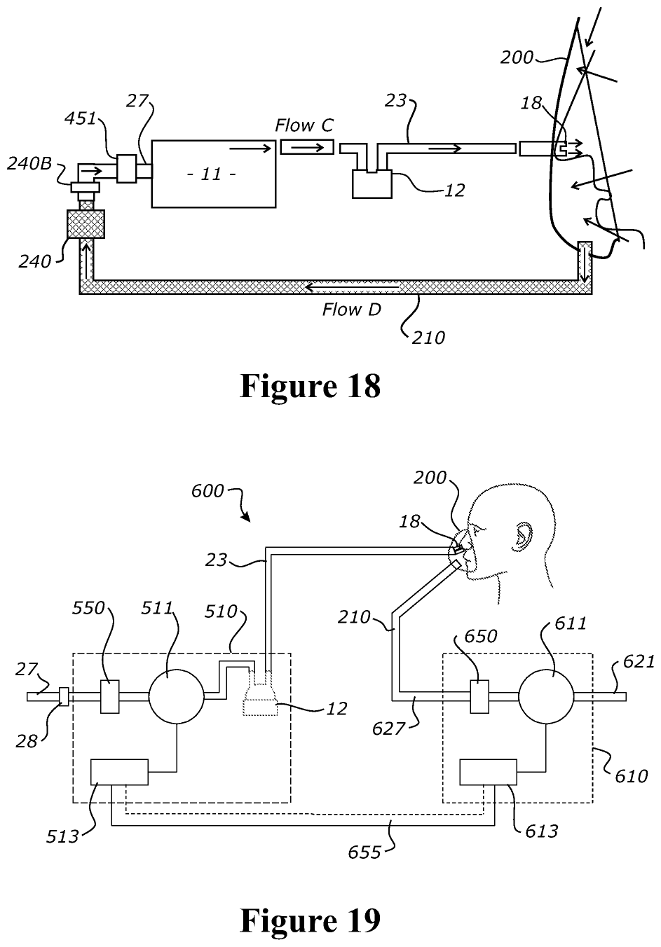

With reference to FIGS. 17 and 18, a system similar to that of FIG. 2 is provided. However, in this embodiment an oxygen concentrator 451 is included in the system. The oxygen concentrator is configured to take ambient air and remove other gases such as nitrogen to create a concentrated oxygen gas. This can be achieved through different methods, such as pressure swing adsorption or membrane gas separation. The oxygen concentrator 451 may be positioned downstream of the CO₂ scrubber 240 and filter, upstream of the flow generator 11. The oxygen concentrator 451 extracts oxygen from the environment and delivers it into the inspiratory circuit, and in this example, into the blower inlet. This results in less oxygen being required from a wall oxygen source or an oxygen tank.

In the embodiment of FIG. 17, the supplementary oxygen supply 28 is retained, with the oxygen concentrator 451 upstream, of the oxygen supply 28. In the embodiment of FIG. 18, the supplementary oxygen supply 28 is omitted, such that any and all oxygen required is provided by the oxygen concentrator 451.

An oxygen concentrator 451 can provide up to approximately 8 l/min of oxygen. A patient uses a relatively negligible amount of oxygen, approximately 0.25 to 0.5 l/min. Thus, if the loss of oxygen at the patient interface 17 can be reduced to less than approximately 8 l/min, the need for a wall or tank oxygen supply is eliminated, as the oxygen concentrator 451 meets the oxygen demand for the system. This is the system shown in FIG. 18. The oxygen concentrator 451 takes in ambient air, filters it through a sieve, releases nitrogen to atmosphere and provides the oxygen from the ambient into the recirculated gases stream. The oxygen concentrator output combined with the oxygen within the recirculated gases can help to increase the concentration of oxygen provided to the inlet of the blower and back to the user.

O2 concentrator can be used instead of a O2 tank or wall O2 since expired/leaked O2 is recirculated and reused.

### Use of Two Flow Generators

In previous embodiments, gases flow delivery via inspiratory conduit 23, and expiratory gases removal via expiratory conduit 210 are provided by the outlet and inlet respectively of a single flow generator 11. With reference to FIGS. 19 to 24 we provide an apparatus in which gases flow delivery via inspiratory conduit 23 is provided by delivery gases flow generator, and expiratory gases removal via expiratory conduit 210 is provided via an expiratory gases flow generator. This apparatus uses two flow generators: one which delivers inspiratory gases flow from the outlet of the first flow generator, and the other of which draws in expiratory gases flow from the inlet of the second flow generator.

Using separate flow generators for these two functions allows the independent control of flow rates. One advantage related to this is that a leak flow between blower outlet and patient interface is not required, as the flow rate of the second blower that applies suction can be increased independently. As such, the suctioning flow rate should be at least 15 L/min higher than the delivered flow rate.

The delivery gases flow generator and the expiratory gases flow generator may be provided in a single apparatus, that is both flow generators are located in a single, common apparatus housing. Alternatively, the delivery gases flow generator may be housed in a delivery apparatus, and the expiratory gases flow generator may be housed in an expiratory or suction apparatus. The delivery apparatus and the expiratory apparatus are separate, and each have separate housings. The delivery and expiratory apparatus may be substantially identical, such that a generic apparatus can be used in a delivery mode as a delivery apparatus, or in a removal mode as a expiratory apparatus. Alternatively the delivery apparatus and the expiratory apparatus can be different. For example, the expiratory apparatus can omit the humidifier at least.

The delivery apparatus and the expiratory apparatus may be configured to communicate with one another, for example via a wired or wireless link. This could allow one of the apparatus to set the operation parameters for both itself and the other apparatus, or vice versa. For example, the expiratory apparatus may be switched on first by a user, and then used to set the parameters and activate the delivery apparatus. Wireless communication may be performed using Bluetooth, infrared, Zigbee, NFC, Wi-Fi, or any other suitable protocol. One apparatus can function as a master apparatus, configured to control the other apparatus as a slave apparatus. Alternatively each apparatus can fully or partially control the other.

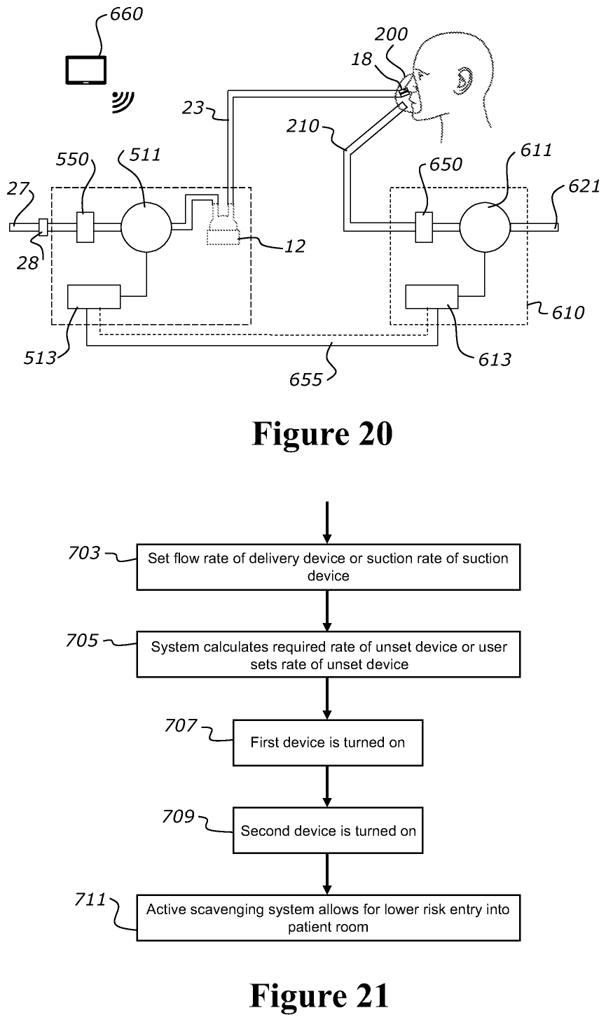

With reference to FIGS. 19 and 20, in this embodiment a respiratory support apparatus 600 is provided comprising a delivery apparatus 510 and a expiratory apparatus 610.

Delivery apparatus 510 is in line with the apparatus 10 of FIG. 1A, with like features being given like references. Delivery apparatus 510 is thus similar to the apparatus of FIG. 2. Thus, delivery apparatus 510 may comprise some or all of the features of the apparatus of FIG. 1 for example. In this example, delivery apparatus 510 comprises a flow generator in the form of a blower 511, a patient or inspiratory conduit 16, and a first patient interface comprising a non-sealing nasal cannula comprising prongs 18, wherein the patient conduit 16 comprises an inspiratory gases flow path between the blower outlet 21 and the patient, configured to deliver a high flow of gases to the patient interface. In this example, the apparatus 10 also comprises humidifier 12. The flow generator 511 in this example comprises a single blower, an inlet comprising ambient air inlet port 27, and outlet port 21. The nasal cannula engages in an unsealed manner with the nostrils of the patient. A supplementary gases inlet 28, for example an oxygen inlet 28, may also be provided. An inlet filter module 550 may also be provided, upstream of the flow generator 511. The filter module 550 removes ambient air particles and/or other particulate matter from the gas to be delivered to the patient.

The delivery apparatus 510 comprises a controller 513 which can control the flow generator 511 to generate a gases flow of the desired flow rate. The controller 513 can also control a supplemental oxygen inlet to allow for delivery of supplemental oxygen, the humidifier 12 (if present) can humidify the gases flow and/or heat the gases flow to an appropriate level, and/or the like. The gases flow is directed out through the patient conduit 16 and cannula 17 to the patient. The controller 513 can also control a heating element in the humidifier 12 and/or the heating element 16a in the patient conduit 16 to heat the gas to a desired temperature for a desired level of therapy and/or level of comfort for the patient. The controller 513 can be programmed with or can determine a suitable target temperature of the gases flow. The controller 513 in this embodiment can also control the expiratory apparatus 610.

The controller 513 can communicate with the expiratory apparatus 610 controller 613 and may also communicate with peripheral devices such as smart phones, tablets, and/or other medical devices. The controller 513 may be compatible with various wired and/or wireless communication protocols such as Bluetooth, NFC, Wi-Fi etc. The controller 513 responds to user and/or system-initiated changes to delivery flow rate and controls the flow generator's 11 motor to rotate at the required angular velocity to produce the specified flow rate.

FIGS. 19 and 20 show a wired (solid line) and/or wireless (dashed line) communication link 655 extending between the apparatus 510, 610. Wired communication link may be supplemented by, or replaced by, a wireless communication link, using suitable transceivers on each apparatus 510, 610. Likewise, reference is made to controller 513 of delivery apparatus 510 controlling expiratory apparatus 610. However, all such references should be considered to include controller 613 of expiratory apparatus 610 controlling delivery apparatus 510. Indeed, it is envisaged that delivery apparatus 510 may be similar or even identical to expiratory apparatus 610, so that multiple identical apparatus can be provided, each configured to operate in a delivery mode or a removal mode as required. Each mode can be configured to activate or deactivate certain features of the apparatus. For example when in removal mode a humidifier of the apparatus may be deactivated.

In accordance with this disclosure, an expiratory flow path (i.e. an expiratory circuit) is provided extending between the face shield 200 and the inlet 27 of a flow generator. However, in this embodiment the expiratory conduit 210 extends to the inlet 627 of flow generator 611 of expiratory apparatus 610. Expiratory apparatus 610 also comprises a flow generator outlet 621, to ambient, and a controller 613. Expiratory apparatus 610 may comprise one filter 650, preferably multiple filters, both upstream and downstream of the flow generator 11. Expiratory apparatus 610 will not require a humidifier, and the filtered, suctioned expiratory gases are delivered to atmosphere, rather than recirculated back into the inspiratory circuit of the apparatus 600. The expiratory apparatus 610 may comprise a humidifier at all, the gases being routed to ambient. Alternatively, in the expiratory apparatus 610 the receptacle of the humidifier may comprise a filter located therein.

The expiratory apparatus 610 includes one or more filter modules within the flow path contained in the apparatus housing. For example, in the embodiments of FIGS. 19 and 20, a single filter module 650 is shown upstream of the flow generator 11. A filter module 650 may contain one or more filters which may be, for example, HEPA filters, pleated filters, virus filters (e.g. PM1 filters), a combination thereof, or any other suitable filter arrangement. The filter module(s) 650 captures aerosols and pathogens e.g. bacteria and viruses. The filter module(s) 650 may comprise a single filter cartridge. The filter cartridge comprises the plurality of filters and is removable from the expiratory apparatus 610 such that it can be replaced.

FIGS. 19 and 20 depict a single filter module 650 located in the pneumatic path in between the inlet 627 of the expiratory apparatus 610 and the flow generator 611. However, multiple filter modules can be used to ensure adequate capturing of expired patient gases/aerosols. Various filter arrangements are possible to prevent, or least minimize, expired patient gases/aerosols from escaping to ambient air such as below:
a) one or multiple filter modules along the expiratory conduit 210 (between facial mask 200 or hood 401, and the expiratory apparatus inlet 627). These filter modules may be external of the housing of the expiratory apparatus 610.
b) one or multiple filter modules between the expiratory apparatus inlet 627 and the expiratory apparatus flow generator 611. These filter modules may be internal of the housing of the expiratory apparatus 610, upstream of the flow generator 611.
c) Having one or multiple filter modules between the expiratory apparatus flow generator 611 and expiratory apparatus outlet 621. These filter modules may be internal of the housing of the expiratory apparatus 610, downstream of the flow generator 611.
d) a combination of the above.

Expired gases exhaled by the patient are forced along the expiratory flow path by the flow generator 11 of the expiratory apparatus 610. The expiratory flow path comprises one or more expiratory ports in the face shield 200 and one or more expiratory conduits 210 and suitable conduit connectors, to connect the expiratory conduit(s) 210 to the expiratory port(s) of face shield 200 and expiratory apparatus flow generator inlet 27. The expiratory conduit 210 may comprise a breathable portion or limb that allows passage of water vapour without allowing passage of liquid water. The flow generator 611 generates the flow required to create a negative pressure pneumatic pathway from the face shield 200 or hood 401, through the expiratory conduit 210, the expiratory apparatus inlet 627, filter arrangement 650, flow generator 611, to the expiratory apparatus outlet 621.

The delivery apparatus 510 and expiratory apparatus 610 can pair with each other to enable communication between the two apparatus 510, 610. This pairing may be a two-way communication channel supported by wired and/or wireless communications link 655. For example, the apparatus 510, 610 may have integrated Bluetooth chips to support Bluetooth communication and/or other hardware to support for example Infrared or UWB. The apparatus 510, 610 may also have physical ports to support I/O for wired connections.

Having such communication in place between the two apparatus 510, 610 allows for the system to provide safety functionality and provide effective patient gases/aerosols extraction. For example, a scenario in which communication between delivery and expiratory apparatus 510, 610 plays a role is the following. The delivery apparatus 510 may transmit an activation signal to the expiratory apparatus 610 when the two are paired and when the delivery apparatus 510 is activated. The delivery apparatus 510 may also transmit the current set flow rate of delivered gases to the expiratory apparatus 610. The expiratory apparatus 610 may then automatically set an expiratory gases removal rate—i.e. by setting the motor speed of its flow generator 611 to create a suction that is a function of the set flow rate of the delivery apparatus' flow generator 511. Communication between the delivery apparatus 510 and expiratory apparatus 610 and more specifically between their controllers 513, 613 is therefore required, to provide a robust expiratory gases extraction system. In this example it is the delivery apparatus 510 that acts as a master apparatus and the expiratory apparatus 610 as a slave, influenced by the delivery apparatus 510 settings and activity. A two-way communication channel 655, however, can accommodate a scenario in with the master/slave roles of the two apparatus 510, 610 are reversed, or are interchangeable. Various procedures for operating the apparatus 600 are explained below with reference to the flow diagrams of FIGS. 21 to 24.

Mobile peripheral devices with support for wireless communication can be paired and used with the system. This is to enable users of the apparatus 600 such as doctors, nurses, and clinicians to operate the system from a safe distance (e.g. outside of the patient's room). This may be desirable when the patient has a contagious condition which can spread through their expired gases/aerosols. In such situations, an operator of the apparatus 600 may want to ensure that effective expiratory gas scavenging is active before they enter the patient's room and come into close proximity with the patient. This would otherwise put them at risk of exposure to harmful matter in the air if an effective scavenging system is not in place. Mobile communication allows for apparatus configuration and activation at a safe distance which would be desirable in a hospital setting.

FIG. 20 shows a system comprising the delivery apparatus 510 and expiratory apparatus 610 of FIG. 19, configured to communicate wirelessly with a remote peripheral mobile device 660, such a remote computer, laptop, tablet or smart phone. In particular the controllers 513, 613 may be configured to enable such communication, and to receive control signals from the peripheral device 660, the control signals being processed by one or both controllers 513, 613, to control one or both apparatus 510, 610.

As described above, the apparatus 600 includes a face shield 200, 401 face shield 200, 401, to ‘trap’ patient expired gases/aerosols within the immediate area around the patient's face. This area can then be vacuumed by the expiratory apparatus 610 via the expiratory conduit 210, as patient expired gases/aerosols are sucked from the face shield 200, 401 into the expiratory conduit 210. The connection between the face shield 200, 401 and the expiratory conduit 210 should be appropriately sealed to prevent, or at least minimise, patient expired gases/aerosols from escaping into ambient air.

It is envisaged that the face shield 200, 401 is arranged such that patient expired gases/aerosols are trapped regardless of the orientation of the patient's head, for example regardless of whether the patient is lying flat or if their head is on its side during side sleeping for example. As described in more detail above, the face shield 200, 401 may take the form of a face shield 200, complete dome or hood 401, a smaller face mask that covers the face, mouth, nose, or a combination thereof, or a total face mask that covers the mouth nose and eyes of the patient.

The face shield 200, 401 is not sealed to the patient's face so as to avoid creating a pressure that may put the patient at risk. This allows ambient air to be sucked through the face shield 200, 401's containment area and through the expiratory conduit 210 to the expiratory apparatus 610. This is important as the expiratory apparatus 610 will be running at a higher flow rate than the delivery apparatus 510, and thus the apparatus 600 requires ambient air to freely flow into the expiratory flow path.

The suction rate of the expiratory apparatus 610 is greater than the set flow rate of the delivery apparatus 510. The suction rate may be at least 15 L/min greater than the set flow rate of the delivery apparatus 510, and may be 20 L/min greater. Alternatively, the suction rate is greater by a specific mathematical relationship or ratio, for example twice the delivery apparatus flow rate. This relationship between the delivery flow rate and suction rate ensures substantially all, or at least sufficient, expired gases/aerosols are drawn from the face shield 200, 401 through the filter arrangement 650 in the expiratory apparatus 610 where any harmful aerosols are collected, contained, and may be disposed of.

In an alternative embodiment to that of FIG. 20, the face shield 200, 401 may be omitted and the inlet end of the expiratory conduit 210 held in place adjacent the patient's face by a mount, such as the strap 211 of FIG. 12 or the collar 213 of FIG. 13.

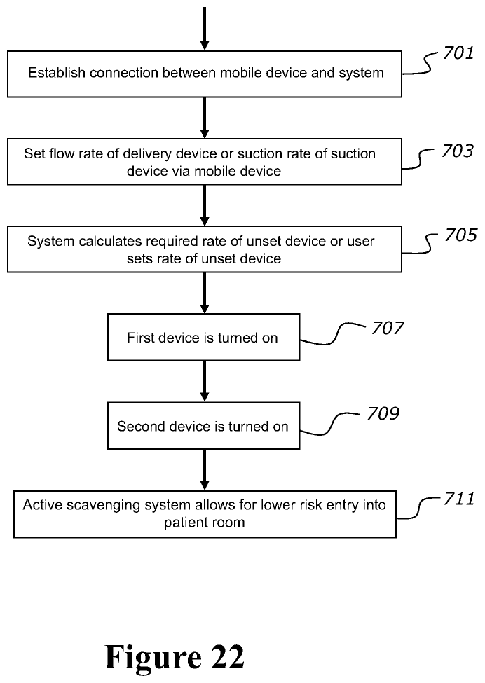

With reference now to FIGS. 21 and 22, these figures are control diagrams showing examples of how the system of FIGS. 19 and 20 are set-up, activated, configured and/or altered during its active state.

With reference initially to FIG. 21, the process involves an initial step 703 of a doctor or clinician (user) manually setting either or both of the flow rates of the delivery apparatus 510 and expiratory apparatus 610. For example, the user sets the delivery apparatus 510 flow rate or the expiratory apparatus 610 flow rate manually either by interacting with the apparatus interface(s) or by using a mobile peripheral device 660. At step 705, the apparatus 600 then automatically sets the flow rate of the other apparatus 510, 610. In the embodiment of FIGS. 20 and 22, the user is able to set both delivery apparatus 510 and expiratory apparatus 610 flow rates manually via the apparatus interfaces and/or through peripheral device 660. If peripheral device 660 is used, an initial control step 701 is provided in which a connection between the peripheral device 660 and one or both apparatus 510. 610 is established. The apparatus 600 may be configured to prevent the user from being able to set flow rates where the expiratory apparatus 610 flow rate is less than a predetermined L/min higher than the delivery apparatus 510 flow rate, for example 15 L/min higher.

At step 707, the user then starts one of the apparatus 510, 610, again manually either by interacting with the apparatus interface(s) or by using a peripheral device 660. That is, the flow generator 511 of one of the apparatus 510, 610 is activated. Preferably, the first apparatus that is started is the expiratory apparatus 610 to ensure that there is active suction at all times during operation and at least before gases are delivered to the patient. This can be achieved by the apparatus 600 preventing the user from starting the delivery apparatus 510 before the expiratory apparatus 610. The user then starts the second apparatus, at step 709, preferably the delivery apparatus 510, once there is active suction. One or both of the controllers 513, 613 may be configured to prevent activation of the second apparatus 510, 610 until the first has been activated. Once both apparatus 510, 610 are activated, step 711 provides active scavenging of expiratory gases from the patient. One benefit of this is that the risk of harmful components being present ion the room air, is reduced, prior to entry into the room by medical personnel.

Some alternatives to the above steps regarding system start after set-up include:
a) The apparatus 600 automatically starts the second apparatus 510, 610 a predetermined amount of time after the first apparatus 510, 610 is started.
b) A user-initiated event occurs (e.g. user-input confirmation of set flow rates). The apparatus 600 them automatically starts the first apparatus 510, 610 after a certain amount of time. After a second period of time apparatus 600 automatically starts the second apparatus 510, 610.
c) The apparatus 600 starts both apparatus 510, 610 concurrently a certain amount of time after a user triggered event (e.g. user-input confirmation of set flow rates for each apparatus 510, 610).

Described above are variations in the sequence in which the apparatus 510. 610 are started. Consistent in each example is that the apparatus 600 ensures that the time period in between the starting of each apparatus 510. 610 (if there is any time period) is short enough to ensure that patient expired gases/aerosols do not escape into ambient room air. The likelihood of patient expired gases/aerosols escaping is lowest when the expiratory apparatus 610 is active before the delivery apparats 510 is turned on, and highest when the delivery apparatus 510 is turned on before the expiratory apparatus 610. The apparatus 600 may still adequately provide effective scavenging of patient expired gases/aerosols if the two apparatus 510. 610 are concurrently started and if the delivery apparatus 510 is the first apparatus activated, provided the expiratory apparatus 610 is turned on before any matter has a chance to escape the face shield 200, 401. The apparatus 600 ensures this delay (if any) is an appropriately safe time period, and that the expiratory apparatus 610 is at the required flow rate to scavenge the patient expired gases/aerosols.

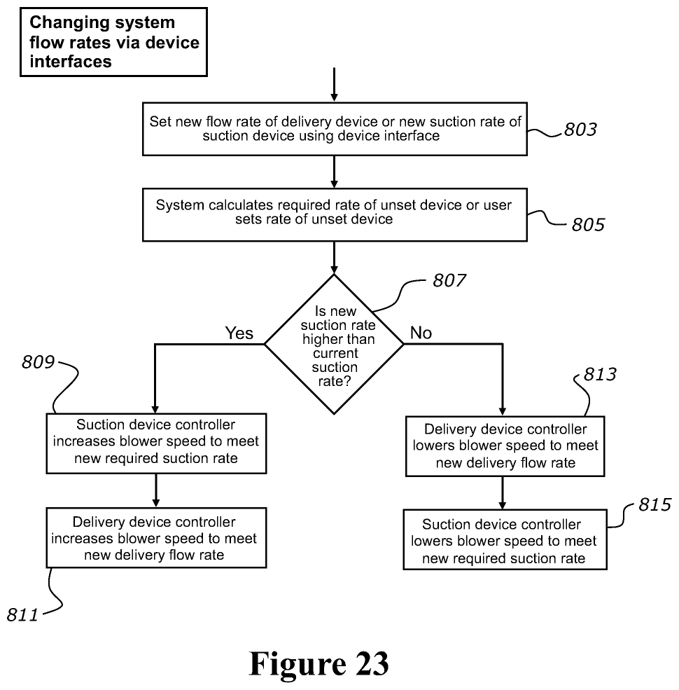

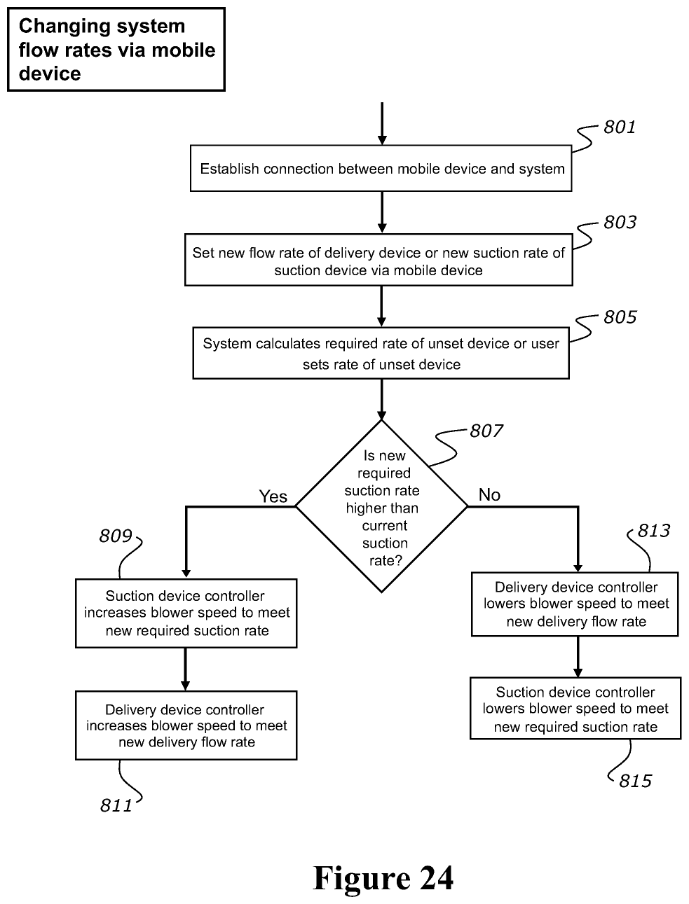

We refer now to FIGS. 23 and 24, which respectively show example control processes for changing system flow rates for the systems of FIGS. 19 and 20.

These control processes involve a doctor or clinician (user) manually changing either or both of the delivery and removal flow rates of the apparatus 600 after the apparatus 600 has already been set-up and started. For example, at a step 803, the user changes the delivery apparatus flow rate or the expiratory apparatus 610 flow rate manually either by interacting with the apparatus interface(s) or by using a peripheral device 660. At step 805, the apparatus 600 then automatically calculates and sets the flow rate of the other apparatus 510, 610. In the embodiment of FIGS. 20 and 24, the user is able to change both delivery apparatus 510 and expiratory apparatus 610 flow rates manually via the device interface(s) and/or through a peripheral device 660. In this embodiment, an initial step 801 comprises establishing wireless communication between the peripheral device 660 and one or both controllers 513. 613.

At step 807, the apparatus 600 should prevent the user from being able to set flow rates where the expiratory apparatus 610 flow rate is less than a predetermined flow rate higher than the delivery apparatus 510 flow rate, for example 15 L/min.

If it is determined at step 807 that the expiratory gases removal flow rate is greater than the current inspiratory gases delivery flow rate, the controllers 513, 613 then, at steps 809, 811 change the flow rates of the apparatus 510, 610 after a certain period of time or after a user triggered event (e.g. user-input confirmation of set flow rates). This should be done in a way to ensure the predetermined higher flow rate of the expiratory apparatus 510 is maintained. The flow rate of the expiratory apparatus 610 may therefore be increased at step 809, and/or the flow rate of the delivery apparatus 510 may be decreased at step 811.

If it is determined at step 807 that the expiratory gases removal flow rate is not greater than the current inspiratory gases delivery flow rate, the flow rate of the delivery apparatus 510 may therefore be increased at step 813, and/or the flow rate of the expiratory apparatus 610 may be decreased at step 815. The flow rates of both apparatus 510, 610 can begin to change concurrently, or sequentially. Once the predetermined difference between the delivery and removal flow rates is achieved, one or both controller 513, 613 monitor that difference and are configured to maintain that difference as closely as possible. In the case that the delivery apparatus 510 flow rate is increased such that it becomes less than 15 L/min lower than the expiratory apparatus 610 flow rate, the period of time this is true should be minimised. The same safe time period considerations as mentioned above apply.

Reference is made above to the desirability of ensuring that the expiratory gases removal flow rate (as determined by the speed of the impeller of expiratory apparatus flow generator 511) is a predetermined amount higher than the inspiratory gases delivery flow rate (as determined by the speed of the impeller of delivery apparatus flow generator 611). In the embodiments above, an example predetermined amount of 15 L/min is given. The predetermined amount could be between 1 and 30 L/min, preferably 5 and 25 L/min, more preferably between 10 and 20 L/min, and most preferably between 13 and 17 L/min. In some embodiments the predetermined amount is 15 L/min or more.

An apparatus 600 comprising deliver apparatus 510 and expiratory apparatus 610 does not require a leak flow from the inspiratory circuit. Consequently, leak flow port or valve 230 is not required.

Such an apparatus 600 enables independent control of the inspiratory gases flow rate to the patient and the expiratory gases flow rate from the patient.

The outlet end of expiratory conduit 210 is connected to the inlet of the second apparatus, that is, the expiratory apparatus 610.

The delivery apparatus 510 and expiratory apparatus 610 may be identical. The delivery apparatus 510 and expiratory apparatus 610 may be different. He expiratory apparatus 610 may omit a humidifier. The removal device 610 may be provided with one or more filters, or additional filters, as described above, to filter aerosols and undesirable components from the expiratory gases flow, prior to the flow being delivered or vented to atmosphere.

The apparatus 600 comprising the delivery apparatus 510 and expiratory apparatus 610 may further comprise a patient interface comprising a non-sealing nasal cannula. The apparatus 600 may be configured as a high flow apparatus, for delivering nasal high flow therapy to the patient.

Reference to any prior art in this specification is not, and should not be taken as, an acknowledgement or any form of suggestion that the prior art forms part of the common general knowledge in the field of endeavor in any country in the world.

Where reference is used herein to directional terms such as ‘up’, ‘down’, ‘forward’, ‘rearward’, ‘horizontal’, ‘vertical’ etc., those terms refer to when the apparatus is in a typical in-use position, and are used to show and/or describe relative directions or orientations.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The terms “approximately,” “about,” and “substantially” as used herein represent an amount close to the stated amount that still performs a desired function or achieves a desired result. For example, in some embodiments, as the context may permit, the terms “approximately”, “about”, and “substantially” may refer to an amount that is within less than or equal to 10% of, within less than or equal to 5% of, and within less than or equal to 1% of the stated amount.

Reference to any prior art in this specification is not, and should not be taken as, an acknowledgement or any form of suggestion that that prior art forms part of the common general knowledge in the field of endeavour in any country in the world.

The disclosed apparatus and systems may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

Depending on the embodiment, certain acts, events, or functions of any of the algorithms, methods, or processes described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially.

It should be noted that various changes and modifications to the presently preferred embodiments described herein will be apparent to those skilled in the art. Such changes and modifications may be made without departing from the spirit and scope of the disclosed apparatus and systems and without diminishing its attendant advantages. For instance, various components may be repositioned as desired. It is therefore intended that such changes and modifications be included within the scope of the disclosed apparatus and systems. Moreover, not all of the features, aspects and advantages are necessarily required to practice the disclosed apparatus and systems. Accordingly, the scope of the disclosed apparatus and systems is intended to be defined only by the claims that follow.

1. A respiratory support apparatus configured to provide a gases flow to a patient, the respiratory support apparatus comprising:
a flow generator configured to generate the gases flow and comprising a flow generator inlet and a flow generator outlet, the flow generator being configured to draw inspiratory gases in through the flow generator inlet, and to deliver the gases flow through the flow generator outlet;
a patient interface configured to be mounted on the patient and to receive the gases flow from the flow generator outlet to deliver the gases flow to the patient;
an expiratory conduit configured to extend between, and be in fluid communication with, a region at or adjacent the patient interface and the flow generator inlet, to provide a flow path for expiratory gases between the region at or adjacent the patient interface and the flow generator inlet;
wherein the generation by the flow generator of the gases flow also generates forces in the expiratory conduit, such that the flow generator forces expiratory gases along the expiratory conduit from the region at or adjacent the patient interface; and
wherein the respiratory support apparatus comprises a leak port or a valve configured to provide a controlled leak of the gases flow between the flow generator outlet and the patient interface.

2. The respiratory support apparatus of claim 1, wherein the leak port or the valve is provided at the flow generator outlet at a location that is upstream of the patient interface.

3. The respiratory support apparatus of claim 2, wherein the patient interface comprises a nasal cannula or a sealed mask, and the leak port or the valve is provided at the nasal cannula or the sealed mask.

4. The respiratory support apparatus of claim 3, wherein the leak port or the valve is configured to leak between 5 and 50% of the gases flow.

5. The respiratory support apparatus of claim 2, wherein the leak port or the valve is configured to leak a predetermined flow rate of the gases flow.

6. The respiratory support apparatus of claim 5, wherein the predetermined flow rate of the gases flow is between 15 l/min and 25 l/min.

7. The respiratory support apparatus of claim 2, wherein the leak port or the valve is configured to leak a predetermined proportion of the gases flow.

8. The respiratory support apparatus of claim 2, wherein the leak port or the valve is configured to vary an amount of the gases flow that leaks through the leak port or the valve.

9. The respiratory support apparatus of claim 1, wherein the flow generator inlet comprises an ambient inlet configured to receive ambient air.

10. The respiratory support apparatus of claim 9, wherein the flow generator inlet comprises a supplemental gases inlet configured to receive supplemental gases.

11. The respiratory support apparatus of claim 10, wherein the supplemental gases comprise concentrated oxygen.

12. The respiratory support apparatus of claim 1, wherein the flow generator is configured to recirculate expiratory gases received from the expiratory conduit into the gases flow.

13. The respiratory support apparatus of claim 1 further comprising a filter that is located between the patient interface and the flow generator and that is configured to filter the expiratory gases.

14. The respiratory support apparatus of claim 1 further comprising a gases scrubber that is located between the patient interface and the flow generator and that is configured to scrub the expiratory gases.

15. The respiratory support apparatus of claim 14, wherein the gases scrubber is a CO2 scrubber.

16. The respiratory support apparatus of claim 1 further comprising an inspiratory conduit configured to provide an inspiratory gases flow path for the gases flow between the flow generator outlet and the patient interface.

17. The respiratory support apparatus of claim 16 further comprising a humidifier that is configured to humidify the gases flow.

18. The respiratory support apparatus of claim 17, wherein the leak port or the valve is located upstream of the humidifier.

19. The respiratory support apparatus of claim 17, wherein the leak port or the valve is located downstream of the humidifier.

20. The respiratory support apparatus of claim 1 further comprising a plurality of leak ports or valves.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)
